(19) United States
(12) Patent Application Publication
(10) Pub. No.: US 2006/0115480 A1
(43) Pub. Date: Jun. 1, 2006

(54) Disease treatment via antimicrobial peptide inhibitors

(76) Inventor: Yitzchak Hillman, Jerusalem (IL)

Correspondence Address: Martin Moynihan;c/o Anthony Castorina, 2001 Jefferson Davis Highway, Arlington, VA, 22202, US

(21) Appl. No.: 10/539,558

(22) PCT Filed: Dec. 21, 2003
(86) PCT No.: PCT/IL03/01094

§ 371 Date: Jun. 17, 2005

(30) Foreign Application Priority Data
Dec. 19, 2002 (IL) 153557
Jul. 17, 2003 (IL) 156980

(51) Int. Cl. A61K 48/00; C07K 16/40; A61K 39/395

(52) U.S. Cl. 424/146.1; 514/44; 530/388.26

(57) ABSTRACT

A method of treating a disease in a subject in need thereof is disclosed. The method comprises providing to the subject a therapeutically effective amount of a compound being capable of decreasing an activity and/or level of an antimicrobial peptide (AMP) and/or AMP-like molecule, thereby treating the disease in the subject in need thereof.

### FIELD AND BACKGROUND OF THE INVENTION

The present invention relates to methods of treating diseases using anti-antimicrobial peptide (AMP) and/or anti-AMP-like molecule (AML) inhibitors, to methods of treating diseases using AMPs/AMLs, and to methods of identifying compounds capable of decreasing activities/levels of AMPs/AMLs so as to enable treatment of diseases. More particularly, the present invention relates to methods of treating diseases, including autoimmune diseases such as psoriasis and malignancies such as carcinomas, which are associated with inflammation, dysregulated cell proliferation/differentiation, angiogenesis and/or metastasis by using compounds capable of inhibiting levels/activity of cathelicidins/beta-defensins; to methods of treating diseases, such as epithelial wounds, which require therapeutic stimulation of epithelial proliferation, by using beta-defensins; and to methods of identifying compounds capable of decreasing activity/levels of AMPs so as to enable treatment of diseases including autoimmune diseases such as, psoriasis and malignant diseases such as carcinomas, which are associated with inflammation, dysregulated cell proliferation/differentiation, angiogenesis and/or metastasis.

Diseases, such as malignant, autoimmune, allergic, and wound-associated diseases, which are associated with biological processes such as inflammation, dysregulated cell proliferation/differentiation, dysregulated cell proliferation/differentiation balance, angiogenesis, and metastasis, include a vast range of highly debilitating and/or lethal pathologies, and pathologies of great economic impact, for which no satisfactory treatment methods are presently available.

For example autoimmune diseases represent diseases of major clinical and economic impact. These include major diseases such as psoriasis, rheumatoid arthritis, type I diabetes, inflammatory bowel diseases, and multiple sclerosis for which no satisfactory treatment methods are available. Similarly, malignant diseases, such as skin carcinoma, breast carcinoma, colon carcinoma, head and neck carcinoma, hepatic carcinoma, lung carcinoma, renal cell carcinoma, urinary bladder carcinoma, and the like, represent numerous lethal diseases for which no satisfactory treatment methods are available. Diseases associated with epithelial wounds, include major diseases, such as peptic ulcers, ulcerative colitis, and wound-healing deficiencies such as diabetes related skin ulcerations, which are of great clinical and economic impact and for which no satisfactory treatment methods are available. Allergic diseases, such as allergy to seasonal pollens, ragweed, dust mites, pet fur, cosmetics, and various foods are significantly debilitating to a large proportion of the population, can be fatal, and are of great economic significance due to the large market for allergy drugs.

There is therefore an urgent and long-felt need for optimal methods of treating such diseases which are associated with inflammation, dysregulated cell/tissue proliferation/differentiation, dysregulated cell/tissue proliferation/differentiation balance, angiogenesis metastasis, and/or epithelial wounds.

The epithelial lining of the skin, gastrointestinal tract and bronchial tree produces a number of peptides with antimicrobial activities termed antimicrobial peptides (AMPs), which appear to be involved in both innate host defense and adaptive immune responses (Yang D. et al., 2001. Cell Mol Life Sci. 58:978-89). AMPs are cationic peptides which display antimicrobial activity at physiological concentrations under conditions prevailing in the tissues of origin. AMP synthesis and release is regulated by microbial signals, developmental and differentiation signals, cytokines and in some cases neuroendocrine signals in a tissue-specific manner. Their mode of action is unknown, however the leading theory claims that permeabilization of target membranes is the crucial step in AMP-mediated antimicrobial activity and cytotoxicity. Defensins are classified into two major groups in humans; cathelicidins and defensins. AMPs appear to have common characteristics that enable them to affect mammalian cells in a way that does not necessarily function through a ligand-receptor pathway, and that, being small, and highly ionic or hydrophobic or structurally amphiphilic, AMPs can bind mammalian cell membranes. They are able to penetrate penetrate through the cell membrane to the cytoplasm. For example, it was shown that granulysin penetrates and damages human cell membranes dependent upon negative charge (J. Immunol., 2001, 167:350-356). At high concentrations they are cytotoxic to cells, they tear through the membrane causing lysis or apoptosis. Likewise they are able to change the charge density of the inner membrane by the very fact that they have charge, are small and are distributed around the cell membrane from the outer surface of the membrane.

Cathelicidins contain a conserved “cathelin” precursor domain. Their organization includes an N-terminal signal peptide, a highly conserved prosequence, and a structurally variable cationic peptide at the C-terminus. The prosequence resembles cathelin, a protein originally isolated from porcine neutrophils as an inhibitor of cathepsin L (hence, the name cathelin). The 37 amino acid-long human cathelicidin, LL-37/hCAP18 has a hydrophobic N-terminal domain in an α-helical conformation, particularly in the presence of negatively charged lipids. In a step essential for its activation, LL-37 is enzymatically cleaved from the C-terminus of hCAP18 precursor via enzymes such as neutrophil elastase and proteinase 3. LL-37 functions in synergy with other AMPs, and can directly activate host cells. The ability of cathelicidins such as LL-37 to both kill bacteria and regulate immune responses is a characteristic of numerous AMPs. The peptide can influence host immune responses via a variety of cellular interactions, for example, it has been suggest to possibly function as a chemoattractant by binding to formyl-peptide-receptor-like-1 (FPRL-1). LL-37 can recruit mast cells, then be produced by the mast cell to kill bacteria.

Defensins represent a large family of AMPs which contribute to the antimicrobial action of granulocytes, mucosal host defence in the small intestine and epithelial host defence in the skin and elsewhere (Ganz T. 2003 Nat Rev Immunol. 3:710-20). Defensins are thought to contribute to host defense by disrupting the cytoplasmic membrane of microorganisms. Defensins are produced by the epithelial cell lining of the gastrointestinal and genitourinary tracts, the tracheobronchial tree, and keratinocytes as well as by phagocytic cells and lymphocytes. Some defensins are produced constitutively, and others are produced in response to proinflammatory cytokines and microbial products. Defensins produced during innate host defence serve as signals which initiate, mobilize, and amplify adaptive immune host defenses. These peptides are cationic and include 6-8 cysteine residues forming disulfide bridges. Mammalian defensins can be classified into three distinct sub-families: alpha-defensins, and beta-defensins, as well as the theta-defensins which are absent in humans.

Alpha-defensins have three disulfide bridges in a 1-6, 24, 3-5 alignment. Human neutrophils express four distinct alpha-defensins; alpha-defensin-1 to -4, also referred to as human neutrophil peptides (HNP-1 to -4) which are stored in azurophilic granules of neutrophils as fully processed mature peptides of about 3 kDa. Two additional alpha-defensins, human defensin (HD)-5 and -6 are expressed in small intestinal crypt Paneth cells, and in female urogenital tract epithelial cells. Unlike neutrophils, Paneth cells store alpha-defensins as propeptides. Similarly to cathelicidins, alpha-defensins exert action on both microbes and the host. For example, HNP 1-3 have been shown to increase the expression of tumor necrosis factor (INF)-alpha and interleukin (IL)-1 in human monocytes that have been activated by bacteria (Staphylococcus aureus), or reduce expression of the vascular adhesion molecule-1 (VCAM-1) in human umbilical vein endothelial cells activated by TNF-alpha.

The beta-defensins are characterized by having 6 cysteine motifs connected by three disulfide bridges spaced as C₁-C₅, C₂-C₄, and C₃-C₆. Beta-defensins have been identified numerous cell types, including epithelial cells and neutrophils. Four types are known in humans and are termed human beta-defensin-1, -2, -3 and -4. Genomic analyses suggest that numerous beta-defensins genes remain to be discovered. Beta-defensins display broad spectrum of antimicrobial activity and additional immune cell-related functions. For example, human beta-defensin-2 has the capacity bind the chemokine receptor CCR6, to be chemotactic for dendritic cells and T-cells, and induce histamine release and prostaglandin D2 production in mast cells. Thus, it has been proposed that beta-defensins play a role in allergic reactions. By employing chemokine receptors such as CCR6 on dendritic cells and T cells, defensins may be involved in the modulation of adaptive antimicrobial immunity (Yang D. et al., 1999. Science. 286:525-8; Yang D. et al., 2002. Trends Immunol. 23:291-6; Oppenheim et al., 2003. Ann Rheum Dis. 62 Suppl 2:ii17-21).

AMPs exert their effects either individually or as the resultant effect of multiple AMPs. For example, in the menstrual cycle there is a monthly cycle-dependent expression of various AMPs (King A. E. et al., 2003. J. Reprod. Immunol. 59:1-16). For example, there is higher expression during the menstrual cycle of beta-defensin-2 in the menstrual stage, beta-defensin-4 in the proliferative stage, beta-defensin-3 in the early secretory stage, beta-defensin-1 in the mid secretory stage, and beta-defensin-3 in the late secretory stage. It has been suggested that maintaining the balance between the AMPs is essential for normal proliferation, differentiation and in the specific example of menstrual cycle for development.

Antimicrobial peptide-like molecules (AMLs) such as chemokines, and in particular AMPs that function dually as chemokines or as cytokines, play an important role in orchestrating leukocyte recruitment during inflammation. Monoclonal antibodies and antagonists to chemokines such as for example TNF, IFN-gamma, leukotriene receptor antagonists, IL-8, anti-IgE and anti-IL receptor antagonists are already patented and used clinically. However, these generally have major side effects due to the fact that these chemokines function dually in normal growth and metabolism. Thus, inhibiting their activity also inhibits normal growth of body cells.

However, apart from their antibacterial and anti-viral nature, AMPs play several roles that enhance the pathogenesis of disease. They act individually or in synergy as chemokines, as cytokines, proliferation and hyperproliferation biofilm inducers, bacterial-cellular binding and adhesion enhancers, inflammatory enhancers, indirectly as monocyte iron retention regulators protease inhibitors, angiogenesis enhancers, corticostatin-like molecules, enhance extracellular matrix deposition controlling its degradation, and more. Most importantly, AMPs are upregulated in several chronic diseases, as is detailed below, and play a major role in the pathogenesis of chronic inflammation and disease as well as influencing cellular differentiation and proliferation by various mechanisms.

AMPs generally work downstream (Moon, S K. et al., 2002. Biochim. Biophys. Acta 1590:41-51; King, A E. et al., 2002. Mol. Hum. Reprod. 8:341-349; Seo, S J. et al., 2001. J. Dermatol. Sci. 27:183-191; Tomita, T, and Nagase, T, 2001. Nippon Ronen Igakkai Zasshi 38:440-443) as well as upstream (Harder, J. et al., 2000. Am. J. Respir. Cell Mol. Biol. 22:714-721; Chaly, Y V. et al., 2000. Eur. Cytokine Netw. 11:257-266) to the cytokines and chemokines that are currently inhibited by the current available treatments. They are both activators of the inflammatory reaction as well as being transcribed as a response to pro-inflammatory stimuli such as interleukin 1 alpha (IL-1 alpha), tumor necrosis factor alpha (TNF-alpha) and more.

Due to the dual functionality of AMPs upstream and downstream to cytokines such as TNF-alpha and IL-1, disease states induced by such AMPs enter a self-sustaining cycle of uncontrolled production of TNF-alpha, IL-1, resulting in deteriorating/chronic inflammation. This is especially so in situations where AMPs are overexpressed, to a larger copy number or over-activated polymorphism of genes for AMPs. Breaking of this inflammatory cycle has been achieved using TNF-alpha antagonists, IL-1 receptor antagonists, IL-10 inhibitors, and T-cell inhibitors. However due to the need for inducing minimal of side effects, inhibiting the activity of these AMPs, and in particular, inhibiting their secondary cytokine activity, proves a preferred safer and more effective approach to treatment of inflammatory and autoimmune chronic as well as some acute conditions.

Thus, numerous diseases which are associated with inflammation, dysregulated cell proliferation/differentiation, angiogenesis metastasis, and/or epithelial wounds appear to be associated with dysregulated AMP levels in affected cells/tissues (reviewed, for example, in Gallo and Nizet, 2003. Curr. Allergy and Asthma Reports 3:402; van Wetering et al., 1999. J Allergy Clin Immunol. 104:1131-8).

With regards to psoriasis and other skin pathologies, it has been shown that there are increased levels of LL-37 and beta-defensin-2 in psoriasis lesions, but none to minor amounts in skin from atopic dermatitis patients, with psoriasis patients having at least 10 times as much of such AMPs in their skin as atopic dermatitis patients (Ong P Y et al., 2002. N Engl J Med. 347:1151-60). Furthermore, cutaneous injury, a known psoriasis trigger, induces the release of LL-37 and beta defensin-2, and such injuries may develop into irreversible psoriatic lesions. In addition, there are other skin pathologies associated with increase in AMPs. The majority of acne biopsies display a marked upregulation of defensin-2 immunoreactivity in the lesional and perilesional epithelium—in particular in pustules—and a less marked upregulation of defensin-1 immunoreactivity (Chronnell C M T et al., 2001. J Invest Dermatol 117:1120-1125).

Psoriasis has been established as a T-cell mediated autoimmune disease with innate immunity paying a key role. Psoriasis is a result of a cutaneous defect that is triggered by an autoimmune activation (Gilhar, A. et al., 2002. J. Invest Dermatol. 119:384-391) by bacterial superantigens (Boehncke, W H. et al., 2001. J. Invest Dermatol. 116:596-601). Histologically, psoriasis is characterized by abnormalities in the proliferation/differentiation balance of keratinocytes and fibroblasts, with abnormal differentiation and infiltration of the epidermis and dermis by neutrophils, lymphocytes, macrophages and mast cells. Natural killer (NK) and NK-T cells have been implicated in the pathogenesis of psoriasis and are present in plaques of psoriasis (Br J Dermatol. 2003, 149:160-4). AMPs are effector molecules of human T and natural killer (NK) cells their release from NK cells plays a part in the pathogenesis of disease. The human AMPs LL-37 and alpha-defensins are expressed by specific lymphocyte and monocyte populations (Agerberth B. et al., 2000. Blood. 96:3086-93).

Up to the present, suboptimal novel systemic interventions are used to treat psoriasis. These include mainly T-cell targeted therapies, monoclonal antibody against chemokine tumor necrosis factor and cytokine targeted therapies.

Treatments for psoriasis include topical application of cell proliferation/differentiation regulators such as retinoid—vitamin A—analog, which modulates or changes the cellular differentiation and proliferation of the epidermis by inducing apoptosis thereby limiting the number of proliferations (Ocker, M. et al., 2003. Int. J. Cancer 107:453-459), UV treatment which also induces cell apoptosis thereby reducing the opportunities for cells to proliferate (Mass, P. et al., 2003. Arch. Dermatol. Res. 295:71-79). Apoptosis also enables the release of anionic DNA which forms bundles in the presence of cationic AMPs thereby inhibiting antimicrobial activity and their downstream elements through the ligand-cell receptor connections, corticosteroid creams and ointment and synthetic vitamin D3. These topical treatments are aimed at regulating only the end result inflammation reactivity of the epidermis, they do not prevent the initial process from occurring.

There are many signaling pathways leading to pathogenic proliferation. Abnormality in the proliferation/differentiation balance in psoriasis is a result of overexpression in the AMP pathway on account of other pathways such for example the TGF-beta signaling pathway which is downregulated in psoriatic skin (Doi, H. et al., 2003. J. Dermatol. Sci. 33:7-16), a functional decrease in growth regulation. In fact, it seems that AMPs such as LL-37, human beta-defensin-3, neutrophil gelatinase-associated lipocalin, and secretory leukocyte protease inhibitor act downstream to growth factors important in wound healing such as insulin-like growth factor 1 and TOF-alpha in human keratinocytes (Sorensen, O E. et al., 2003. J. Immunol. 170:5583-5589).

Alpha-defensins have been shown to accumulate in airway secretions of patients with various chronic inflammatory lung disorders, and have been demonstrated to be cytotoxic toward airway epithelial cells and to induce pathogenic chemokine secretion in several cell types. Specifically, alpha-defensins have been shown to be increased in inflamed tissues affected by rhinitis and upper respiratory tract S. aureus infection. Beta-defensins are overexpressed in inflamed sinus fluid of sinusitis patients. Cathelicidin and beta-defensins have been shown to be overexpressed in inflamed bronchi of pneumonia patients. Increased levels of AMPs have been found to correlate with levels of soluble and cellular inflammatory mediators such as IL-8 and neutrophils. Alpha- and beta-defensins have been demonstrated to be expressed at high level in the inflamed respiratory tract of patients infected with Mycobacterium. High levels of defensins have been shown to be associated with damaged tissue in acute respiratory distress syndrome (ARDS), in idiopathic inflammatory lung diseases, such as diffuse panbronchiolitis and in idiopathic pulmonary fibrosis. It has been suggested that neutrophil defensins can induce pathogenic pulmonary epithelial-cell proliferation and incident lung remodeling. Increased levels of AMPs in respiratory tract secretions were shown to correlate with chronic inflammation in cystic fibrosis. Alpha-defensins have been shown to promote bacterial adherence to epithelial cells in vitro suggesting that these peptides play a role in the pathogenesis of diseases such as chronic obstructive pulmonary disease and cystic fibrosis. Increased numbers of neutrophils are also present in the airways of patients with asthma, suggesting that neutrophils are involved in the pathogenesis of this disease. Since defensins have the capacity to induce histamine release by mast cells and thereby increase airway hyperresponsiveness, it is possible that such molecules contribute to asthma pathogenesis. Experiments in mice support the idea that dysregulation of AMP expression may be associated with such disease pathogenesis. For example, intratracheal instillation of defensins was shown to result in acute pulmonary dysfunction, neutrophil invasion, and to bronchial release of inflammatory mediators, such as TNF-alpha and macrophage inflammatory protein (MIP)-2.

With respect to gastrointestinal pathologies, constitutive expression of beta-defensin in inflamed gastric epithelium of patients with gastritis or gastric cancers induced by Helicobacter pylori has been reported. High levels of alpha- and beta-defensins have been observed in the inflamed colonic epithelium of patients suffering from Crohn's disease or active ulcerative colitis.

In the case of urogenital diseases, upregulation of AMP production has been reported in inflamed tissue of urogenital tract infections. Induced expression of beta-defensin-2 has been shown to occur in inflamed tissues in tubulus epithelia with chronic pyelonephritis. Women with pelvic inflammatory diseases secondary to infection with T. vaginalis, N. gonorrhoeae, or Chlamydia trachomatis displayed high neutrophil defensin expression levels in the vagina at levels which were strongly associated with the presence of endometritis.

With respect to malignant diseases, in vitro and in vivo findings suggest that alpha-defensins are frequent peptide constituents of malignant epithelial cells in Renal cell carcinoma with a possible direct influence on tumor proliferation (Muller, C A. et al., 2002. Am. J. Pathol. 160:1311-1324).

Overexpression of AMPs contributing to disease states may occur as a result of several mechanisms, including gene copy number polymorphisms (Hollox, E J. et al., 2003. Am. J. Hum. Genet. 73:591-600), the genomic locations of their promoters, and polymorphisms in these proteins leading to their overexpression or overactivation.

Therefore, in light of the apparent roles for AMPs/AMLs in the pathogenesis of diseases which are associated with inflammation, dysregulated cell proliferation/differentiation, dysregulated cell proliferation/differentiation balance, angiogenesis metastasis, and/or epithelial wounds, the present inventors hypothesized that an optimal strategy for treating such diseases would be via methods involving decreasing the levels/activity of such AMPs/AMLs, and/or via methods involving administering such AMPs/AMLs.

The prior art approaches relating to such methods involve computationally identifying a genetic sequence encoding a novel, putative AMP-like molecule of unknown function, and of unknown relationship to a disease pathogenesis, and proposes attempting to regulate levels of such a molecule for treating a disease (U.S. Pat. Application No. 20030044907).

The prior art approaches, however, are critically flawed. Since a role for the novel putative AMP-like molecule in pathogenesis of any disease is unknown, it cannot be reasonably be expected that regulating the levels of the putative AMP-like molecule will have a therapeutic effect when administered to a subject having a disease. Critically, the prior art approaches have never been attempted, and as such have not demonstrated their viability for treatment of any disease. Importantly, the prior art approaches have not proposed a method of using inhibitors of AMPs such as beta-defensin-2 or LL-37 for treatment any disease.

Thus, the prior art has failed to provide an adequate solution for treating any disease by decreasing levels/activity of an AMP/AMP-like molecule.

There is thus a widely recognized need for, and it would be highly advantageous to have, a method which is devoid of the above limitation for treating diseases associated with inflammation, dysregulated cell proliferation/differentiation, angiogenesis metastasis, and/or epithelial wounds.

### SUMMARY OF THE INVENTION

According to one aspect of the present invention there is provided a method of treating a disease in a subject in need thereof, the method comprising providing to the subject a therapeutically effective amount of a compound being capable of decreasing an activity and/or level of an antimicrobial peptide (AMP) and/or AMP-like molecule, thereby treating the disease in the subject in need thereof.

According to further features in preferred embodiments of the invention described below, administering the compound to the subject is effected by exposing a location of the subject to a carrier which includes the compound at a concentration selected from a range of about 50 nanograms per milliliter to about 1 milligram per milliliter.

According to still further features in the described preferred embodiments, administering the compound to the subject is effected by administering to the subject a plurality of doses of the compound selected from a range of 2 doses to 30 doses, wherein each inter dose interval of the plurality of doses is selected from a range of about 2.4 hours to about 30 days.

According to still further features in the described preferred embodiments, administering the compound to the subject is effected via a route selected from the group consisting of the topical, intranasal, transdermal, intradermal, oral, buccal, parenteral, rectal and inhalation route.

According to still further features in the described preferred embodiments, the disease is associated with a biological process in a cell and/or tissue, wherein the biological process is selected from the group consisting of growth, differentiation, inflammation, metastasis and angiogenesis.

According to still further features in the described preferred embodiments, the subject is human.

According to another aspect of the present invention there is provided an article of manufacture comprising packaging material and a pharmaceutical composition, the article of manufacture being identified for treatment of a disease being associated with a biological process in a cell and/or tissue, the biological process being selected from the group consisting of growth, differentiation, inflammation, metastasis and angiogenesis; the pharmaceutical composition comprising a pharmaceutically acceptable carrier and, as an active ingredient, a compound being capable of decreasing an activity and/or level of an antimicrobial peptide (AMP) and/or AMP-like molecule.

According to further features in preferred embodiments of the invention described below, the pharmaceutically acceptable carrier is selected so as to enable administration of the pharmaceutical composition via a route selected from the group consisting of the topical, intranasal, transdermal, intradermal, oral, buccal, parenteral, rectal and inhalation route.

According to still further features in the described preferred embodiments, the pharmaceutical composition is formulated as a solution, suspension, emulsion or gel.

According to still further features in the described preferred embodiments, the pharmaceutical composition is composed so as to enable exposure of a cell and/or tissue of a subject having the disease to the compound at a concentration selected from a range of about 50 nanograms per milliliter to about 1 milligram per milliliter.

According to still further features in the described preferred embodiments, the pharmaceutical composition is further identified for administration to a subject of a plurality of doses of the pharmaceutical composition selected from a range of 2 doses to 30 doses, wherein each inter dose interval of the plurality of doses is selected from a range of about 2.4 hours to about 30 days

According to still further features in the described preferred embodiments, the cell and/or tissue is selected from the group consisting of an epithelial cell and/or tissue, a skin cell and/or tissue, a keratinocytic cell and/or tissue, a gastrointestinal cell and/or tissue and an endothelial cell and/or tissue.

According to still further features in the described preferred embodiments, the disease is selected from the group consisting of a tumor, an autoimmune disease, an epithelial disease, a skin disease, a gastrointestinal disease, an endothelial disease and a human disease.

According to still further features in the described preferred embodiments, the disease is selected from the group consisting of an epithelial tumor, an epithelial wound, a skin tumor, a skin wound, a gastrointestinal tumor, a gastrointestinal wound, an endothelial tumor, a solid tumor, a metastatic tumor, a skin autoimmune disease, and a malignant tumor.

According to still further features in the described preferred embodiments, the disease is psoriasis or skin carcinoma.

According to yet another aspect of the present invention there is provided a method of regulating a biological process in a cell and/or tissue, the method comprising exposing the cell and/or tissue to a compound being capable of decreasing an activity and/or level of an antimicrobial peptide (AMP) and/or AMP-like molecule, thereby regulating the biological process in the cell and/or tissue.

According to further features in preferred embodiments of the invention described below, exposing the cell and/or tissue to the compound is effected by providing the compound to a subject.

According to still further features in the described preferred embodiments, the providing to the subject the compound is effected by administering the compound to the subject and/or by expressing the compound in the subject.

According to still further features in the described preferred embodiments, the exposing the cell and/or tissue to the compound is effected by exposing the cell and/or tissue to the compound at a concentration selected from a range of about 50 nanograms per milliliter to about one milligram per milliliter.

According to still further features in the described preferred embodiments, the cell and/or tissue is malignant and/or keratinocytic, wherein the exposing the cell and/or tissue to the compound is effected by exposing the cell and/or tissue to the compound at a concentration selected from a range of about 0.4 microgram per milliliter to about 200 micrograms per milliliter, and the AMP and/or AMP-like molecule is a cathelicidin.

According to still further features in the described preferred embodiments, the cell and/or tissue is malignant and/or keratinocytic, wherein the exposing the cell and/or tissue to the compound is effected by exposing the cell and/or tissue to the compound at a concentration selected from a range of about 0.1 microgram per milliliter to about 50 micrograms per milliliter, and the AMP and/or AMP-like molecule is a defensin.

According to still further features in the described preferred embodiments, the cell and/or tissue is a gastrointestinal and/or epithelial cell and/or tissue, wherein the exposing the cell and/or tissue to the compound is effected by exposing the cell and/or tissue to the compound at a concentration selected from a range of about 50 nanograms per milliliter to about 10 micrograms per milliliter, and the AMP and/or AMP-like molecule is a defensin.

According to still further features in the described preferred embodiments, the cell and/or tissue is an endothelial cell and/or tissue, wherein the exposing the cell and/or tissue to the compound is effected by exposing the cell and/or tissue to the compound at a concentration selected from a range of about 50 nanograms per milliliter to about 10 micrograms per milliliter, and the AMP and/or AMP-like molecule is a defensin.

According to still another aspect of the present invention there is provided a method of identifying a compound being capable of regulating a biological process in a cell and/or tissue, the method comprising: (a) exposing the cell and/or tissue to a test compound which is: (i) capable of decreasing an activity and/or level of an antimicrobial peptide (AMP) and/or AMP-like molecule, and/or (ii) the AMP and/or AMP-like molecule; and (b) evaluating a capacity of the test compound to regulate the biological process in the cell and/or tissue, thereby identifying the compound being capable of regulating the biological process in the cell and/or tissue.

According to still further features in the described preferred embodiments, the cell and/or tissue is a cultured cell and/or tissue.

According to still further features in the described preferred embodiments, the cell and/or tissue is derived from a human.

According to still further features in the described preferred embodiments, the exposing the cell and/or tissue to the test compound is effected by providing the test compound to a subject.

According to still further features in the described preferred embodiments, the exposing the cell and/or tissue to the test compound is effected by exposing the cell and/or tissue to a cell which produces the test compound.

According to still further features in the described preferred embodiments, the cell which produces the test compound is a B-cell hybridoma.

According to still further features in the described preferred embodiments, the providing the test compound to the subject is effected by administering the test compound to the subject and/or by expressing the test compound in the subject.

According to still further features in the described preferred embodiments, administering the test compound to the subject is effected via a route selected from the group consisting of the topical, intranasal, transdermal, intradermal, oral, buccal, parenteral, rectal and inhalation route.

According to still further features in the described preferred embodiments, the test compound is selected from the group consisting of: (a) a molecule capable of binding the AMP and/or AMP-like molecule; (b) an enzyme capable of cleaving the AMP and/or AMP-like molecule; (c) an siRNA molecule capable of inducing degradation of an mRNA encoding the AMP and/or AMP-like molecule; (d) a DNAzyme capable of cleaving an mRNA or DNA encoding the AMP and/or AMP-like molecule; (e) an antisense polynucleotide capable of hybridizing with an mRNA encoding the AMP and/or AMP-like molecule; (f) a ribozyme capable of cleaving an mRNA encoding the AMP and/or AMP-like molecule; (g) a non-functional analogue of at least a functional portion of the AMP and/or AMP-like molecule; (h) a molecule capable of inhibiting activation or ligand binding of the AMP and/or AMP-like molecule; and (i) a triplex-forming oligonucleotide capable of hybridizing with a DNA encoding the AMP and/or AMP-like molecule.

According to still further features in the described preferred embodiments, the molecule capable of binding the AMP and/or AMP-like molecule is an antibody or an antibody fragment.

According to still further features in the described preferred embodiments, the antibody fragment is selected from the group consisting of a single-chain Fv, an Fab, an Fab′, and an F(ab′)2.

According to still further features in the described preferred embodiments, the AMP and/or AMP-like molecule is selected from the group consisting of a defensin, a cathelicidin, a cationic peptide, a hydrophobic peptide, a human AMP and a human AMP-like molecule.

According to still further features in the described preferred embodiments, the AMP and/or AMP-like molecule is a beta-defensin.

According to still further features in the described preferred embodiments, the AMP and/or AMP-like molecule is selected from the group consisting of beta-defensin-1, beta-defensin-2 and LL-37.

According to still further features in the described preferred embodiments, the cell and/or tissue is selected from the group consisting of an epithelial cell and/or tissue, a skin cell and/or tissue, a keratinocytic cell and/or tissue, a gastrointestinal cell and/or tissue and an endothelial cell and/or tissue.

According to still further features in the described preferred embodiments, the biological process is selected from the group consisting of growth, differentiation, inflammation, and angiogenesis.

According to a further aspect of the present invention there is provided a method of treating a disease in a subject in need thereof, the method comprising providing to the subject a therapeutically effective amount of an antimicrobial peptide (AMP) and/or AMP-like molecule, thereby treating the disease in the subject in need thereof.

According to further features in preferred embodiments of the invention described below, administering the AMP and/or AMP-like molecule to the subject is effected by exposing a location of the subject to a carrier which includes the AMP and/or AMP-like molecule at a concentration selected from a range of about 2 nanograms per milliliter to about 10 micrograms per milliliter.

According to still further features in the described preferred embodiments, administering the AMP and/or AMP-like molecule to the subject is effected via a route selected from the group consisting of the topical, intranasal, transdermal, intradermal, oral, buccal, parenteral, rectal and inhalation route.

According to still further features in the described preferred embodiments, the subject is human.

According to yet a further aspect of the present invention there is provided an article of manufacture comprising packaging material and a pharmaceutical composition, the article of manufacture being identified for treatment of a disease being associated with a biological process in a cell and/or tissue, the biological process being selected from the group consisting of growth, differentiation, inflammation and angiogenesis; the pharmaceutical composition comprising a pharmaceutically acceptable carrier and, as an active ingredient, an antimicrobial peptide (AMP) and/or AMP-like molecule.

According to further features in preferred embodiments of the invention described below, the pharmaceutically acceptable carrier is selected so as to enable administration of the pharmaceutical composition via a route selected from the group consisting of the topical, intranasal, transdermal, intradermal, oral, buccal, parenteral, rectal and inhalation route.

According to still further features in the described preferred embodiments, the pharmaceutical composition is formulated as a solution, suspension, emulsion or gel.

According to still further features in the described preferred embodiments, the pharmaceutical composition is composed so as to enable exposure of a cell and/or tissue of a subject having the disease to the compound at a concentration selected from a range of about 2 nanograms per milliliter to about 10 micrograms per milliliter.

According to still further features in the described preferred embodiments, the disease is selected from the group consisting of a tumor, an epithelial disease, a skin disease, a gastrointestinal disease and an endothelial disease.

According to still further features in the described preferred embodiments, the disease is selected from the group consisting of an epithelial tumor, an epithelial wound, a skin tumor, a skin wound, a gastrointestinal tumor, a gastrointestinal wound and a malignant tumor.

According to still a further aspect of the present invention there is provided a method of regulating a biological process in a cell and/or tissue, the method comprising exposing the cell and/or tissue to an antimicrobial peptide (AMP) and/or AMP-like molecule, thereby regulating the biological process in the cell and/or tissue.

According to further features in preferred embodiments of the invention described below, exposing the cell and/or tissue to the AMP and/or AMP-like molecule is effected by providing the AMP and/or AMP-like molecule to a subject.

According to still further features in the described preferred embodiments, the providing to the subject the AMP and/or AMP-like molecule is effected by administering the AMP and/or AMP-like molecule to the subject and/or by expressing the AMP and/or AMP-like molecule in the subject.

According to still further features in the described preferred embodiments, the exposing the cell and/or tissue to the AMP and/or AMP-like molecule is effected by exposing the cell and/or tissue to the AMP and/or AMP-like molecule at a concentration selected from a range of about 2 nanograms per milliliter to about 10 micrograms per milliliter.

According to still further features in the described preferred embodiments, the AMP and/or AMP-like molecule is selected from the group consisting of a defensin, a cathelicidin, a cationic peptide, a hydrophobic peptide, a human AMP and a human AMP-like molecule.

According to still further features in the described preferred embodiments, the AMP and/or AMP-like molecule is a beta-defensin.

According to still further features in the described preferred embodiments, the AMP and/or AMP-like molecule is selected from the group consisting of beta-defensin-1, beta-defensin-2 and LL-37.

According to still further features in the described preferred embodiments, the cell and/or tissue is selected from the group consisting of an epithelial cell and/or tissue, a skin cell and/or tissue, a keratinocytic cell and/or tissue and a tumor cell and/or tissue.

According to still further features in the described preferred embodiments, the biological process is selected from the group consisting of growth, differentiation, inflammation and angiogenesis.

According to still further features in the described preferred embodiments, the cell and/or tissue is malignant, wherein the exposing the cell and/or tissue to the AMP and/or AMP-like molecule is effected by exposing the cell and/or tissue to the AMP and/or AMP-like molecule at a concentration selected from a range of about 0.1 microgram per milliliter to about 10 micrograms per milliliter, and the AMP and/or AMP-like molecule is a defensin.

According to still further features in the described preferred embodiments, the cell and/or tissue is a keratinocytic cell and/or tissue, wherein the exposing the cell and/or tissue to the AMP and/or AMP-like molecule is effected by exposing the cell and/or tissue to the AMP and/or AMP-like molecule at a concentration selected from a range of about 2 nanograms per milliliter to about 10 micrograms per milliliter, and the AMP and/or antimicrobial peptide-like molecule is a defensin.

According to still further features in the described preferred embodiments, the cell and/or tissue is derived from a human.

The present invention successfully addresses the shortcomings of the presently known configurations by providing: (i) a method of treating a disease which is associated with a biological process in a cell/tissue such as growth, differentiation, inflammation, metastasis and/or angiogenesis by using a compound which is capable of decreasing levels/activity of an AMP and/or an AMP-like molecule; and/or by using an AMP and/or an AMP-like molecule; (ii) an article of manufacture including such a compound and being labeled for treatment of such a disease; and (iii) a method of identifying such a compound.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a series of photomicrographs depicting stimulation of significant proliferation of malignant keratinocytes by the AMPs human beta-defensin-1 and human beta-defensin-2. Cultured human keratinocytes (HaCaT, clone 6, A-5,1-5, 114 and RT-3) were plated in 24 well dishes at 50,000 cells per plate. After attachment, human beta-defensin-1 or human beta-defensin-2 at a final concentration of 1 microgram per ml was added to the culture medium, the cells were further incubated for 48 hours, and photographs of representative fields were taken (×20).

FIG. 2 is a histogram depicting significant inhibition of growth of malignant human keratinocytes by 1.0 microgram/ml of anti-human beta-defensin-2 antibody. Cultured immortalized, moderately malignant or highly malignant human keratinocytes (HaCaT, A-5, and RT-3, respectively) were plated, allowed to attach, incubated in the presence of anti-human beta-defensin-2 antibody at a concentration of 1.0 microgram/ml for 48 hours, and cell proliferation was estimated via [3(H)]-thymidine incorporation assay.

FIG. 3 is a histogram depicting concentration-dependent positive and negative regulation of growth by anti-LL-37 and anti-human beta-defensin-2 antibody in primary skin keratinocytes. Cultured keratinocytes were treated for 48 hours with antibody against LL-37 (blue bars) at concentrations of 4 (“1×”) or 20 (“5×”) micrograms/ml, or with anti-human beta-defensin-2 antibody (yellow bars) at concentrations of 1 (“1×”) or 5 (“5×”) micrograms/ml. Cell proliferation was estimated by measuring [3(H)]-thymidine incorporation and expressed as percent of control untreated cells. A representative experiment is shown. Each bar represents the mean±SE of triplicates.

FIGS. 4a-c are photomicrographs depicting correction of AMP-induced dysregulation of skin differentiation by anti-AMP (human beta-defensin-2) antibody in a three-dimensional organotypic in-vitro skin model. FIG. 4a depicts results obtained with an untreated control, FIG. 4b depicts results following treatment with human beta-defensin-2, and FIG. 4c depicts results following treatment with anti-human beta-defensin-2 antibody. Twenty-four hours following seeding of the murine epidermal compartment with non-malignant HaCaT human keratinocytes, anti-human beta-defensin-2 antibody at a concentration of 1 microgram/ml, or human beta-defensin-2 at a concentration of 20 ng/ml was added to the growth medium, as indicated. An equal volume of 0.1% BSA were added as control. The cocultures were treated every 2-3 days and after 2 weeks were harvested, fixed in 4% paraformaldehyde, paraffin-embedded and sectioned (6 microns). Sections were stained with hematoxylin and eosin (H&E) following standard procedures. Shown are bright field photomicrographs of representative fields recorded using an Olympus light microscope.

FIGS. 5a-d are photographs depicting efficient treatment of psoriatic skin lesions by anti-LL-37 antibody. FIGS. 5a and 5b depict an untreated control lesion, and an anti-LL-37 antibody-treated lesion prior to treatment on Day 0, respectively. FIGS. 5c and 5d depict untreated control lesion, and anti-LL-37 antibody-treated lesion on Day 3. Note absence of flaking in the antibody treated lesions, indicating correction of skin cell/tissue proliferation/differentiation imbalance.

FIG. 6 is a histogram depicting significant concentration-dependent negative or positive regulation of gastrointestinal epithelial cell proliferation by antibody specific for human beta-defensin-2. Cultured Caco2 human gastrointestinal epithelial cells were treated for 48 hours with anti-human betadefensin-2 antibody at 0.5 microgram/ml (blue/pale bars) or at 1.0 microgram/ml (red/dark bars). Cell proliferation was estimated by measuring [3(H)]-thymidine incorporation and is expressed as percent of control untreated cells. A representative experiment is shown. Each bar represents the mean±SE of triplicates.

FIG. 7 is a histogram depicting significant inhibition of primary endothelial cell proliferation by anti-human beta-defensin-2 antibody. Bovine primary endothelial cells were treated for 48 hours with anti-human beta-defensin-2 antibody at 0.5 microgram/ml (blue/pale bars) or at 1.0 microgram/ml (red/dark bars). Cell proliferation was estimated by measuring [3(H)]-thymidine incorporation and was expressed as percent of control untreated cells. A representative experiment is shown. Each bar represents the mean±SE of triplicates.

### DESCRIPTION OF THE PREFERRED EMBODIMENTS

The present invention is of methods of using compounds capable of decreasing activities/levels of antimicrobial peptides (AMP)/antimicrobial peptide-like molecules (AMLs) and/or of using AMPs/AMLs for regulating in cells/tissues biological processes such as growth, differentiation, growth/differentiation balance, inflammation, metastasis and angiogenesis; of methods of using such molecules for treating diseases associated with such biological processes and/or which are amenable to treatment via regulation of such biological processes; of articles of manufacture which include such molecules and which are labeled as being for use in treating such diseases; and of methods of identifying such compounds capable of decreasing activities/levels of AWs/AMLs and/or of identifying such AMPs/AMLs. Specifically, the present invention can be used to optimally treat a vast range of diseases associated with such biological processes, including inflammatory diseases/diseases associated with cellular proliferation/differentiation imbalance such as psoriasis, diseases associated with wounds, and tumors such as metastatic/malignant carcinomas.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Diseases which are associated with inflammation, dysregulated cell/tissue proliferation/differentiation, dysregulated cell/tissue proliferation/differentiation balance, angiogenesis, and/or metastasis include a multitude of diseases which are of great medical and/or economic impact and for which no satisfactory treatment methods are available. While conceiving the present invention, the present inventors have hypothesized that AMPs/AMLs are involved in the pathogenesis of such diseases, and/or and hence that methods of decreasing activities/levels and/or administering such molecules could be used for treating such diseases.

The prior art approach relating to such methods involves computationally identifying a genetic sequence encoding a novel putative AMP-like molecule of unknown function, and of unknown relationship to a disease pathogenesis, and proposes a highly speculative and theoretical method for attempting to use or regulate such a molecule for treating a disease (U.S. Pat. Application No. 20030044907).

The prior art approach, however, is critically flawed. Since no function is known for the putative AMP-like molecule with respect to the pathogenesis of any disease, it cannot be reasonably be expected that regulating the levels of the putative AMP-like molecule will have a therapeutic effect when administered to a subject having a disease. Hence, the prior art approach is restricted to a theoretical method of using or regulating such a putative AMP-like molecule to treat a disease. The prior art approach does not provide, nor does it find any in other prior art document, any experimental support for the disease treatment method which it proposes. As such the prior art approach does not teach treatment of a specific disease, such as an inflammatory disease or a tumor, nor does it provide the ordinarily skilled artisan with any motivation to treat diseases using a method of the present invention. Furthermore, the prior art approach has not proposed a method of using inhibitors of classical AMPs such as beta-defensin-2 or LL-37 for treatment any disease.

Another prior art approach proposes treating a disease by using an antisense polynucleotide complementary to such a genetic sequence, and administering such antisense polynucleotides according to theoretical regimens so as to treat a disease.

The prior art approaches, however, present critical disadvantages, including: (i) use of highly speculative assignment of the role of such putative AMP-like molecules in disease pathogenesis; (ii) requirement for use of unreliable antibody generation methods; (iii) use of theoretical administration regimens for putative therapeutic agents; and (iv) never having been attempted, and hence not having demonstrated any potential for therapeutic applications.

Thus, the prior art fails to provide a viable method which comprises regulating levels/activities of an AMP/AMP-like molecule for treating a disease.

While reducing the present invention to practice it was uncovered that anti-AMP antibodies could be used to: significantly inhibit growth and loss of substrate attachment of cultured human malignant carcinoma cells; significantly inhibit/induce growth of cultured primary human keratinocytes; efficiently correct human epithelial cell/tissue proliferation/differentiation imbalance in a three-dimensional organotypic cultured skin model; efficiently treat psoriasis in a human subject; significantly inhibit/induce growth of cultured human gastrointestinal epithelial cells; and efficiently inhibit growth of human endothelial cells.

While reducing the present invention to practice, it was also uncovered that AMPs could be used to significantly upregulate or downregulate growth of cultured human epithelial cells.

Hence, in sharp contrast to prior art techniques, the method according to the present invention enables use of compounds capable of decreasing levels/activity of AMPs/AMLs, and/or the use of AMPs/AMLs for regulating biological processes such as growth, differentiation, inflammation, metastasis and angiogenesis, and treatment of numerous diseases, such as those which are associated with inflammation, dysregulated cell proliferation/differentiation, angiogenesis, and/or metastasis, including carcinomas such as malignant metastatic skin carcinomas, wound-associated diseases such as ulcerative diseases, and autoimmune diseases/diseases associated with dysregulated cellular proliferation/differentiation such as psoriasis.

Thus, the present invention provides a method of regulating a biological process in a cell and/or tissue. The method is effected by exposing the cell and/or tissue to: a compound being capable of decreasing an activity and/or level of an antimicrobial peptide (AMP) and/or AMP-like molecule (AML); to an AMP; and/or to an AML.

The method can be used to regulate in a cell/tissue a biological process such as growth, differentiation, inflammation, metastasis and/or angiogenesis. By virtue of enabling regulation of such a biological process in a cell/tissue, the method can be used for treating a disease which is associated with such a biological process, and can be used for identifying the regulator, as described in further detail hereinbelow. Diseases associated with such biological processes include, for example, autoimmune diseases, diseases associated with dysregulated cell/tissue growth/proliferation balance, wound-associated diseases, and tumors.

As used herein, the term “regulator” refers to the compound which is capable of decreasing an activity and/or level of an AMP/AML, to an AMP, and/or to an AML which is used for practicing any aspect of the present invention.

As used herein, the phrases “the compound”, “compound of the present invention”, and “AMP/AML inhibitor” interchangeably refer to the compound which is capable of decreasing an activity/level of an AMP/AML.

Any of various types of AMP/AML inhibitors may be employed according to the teachings of the present invention for regulating the biological process, depending on the application and purpose.

As used herein, the term “AMP” includes any defensin, cathelicidin, and/or thrombocidin, or variant thereof, including any naturally occurring variant of such a molecule, such as a natural mutant/polymorphic variant/allele of such a molecule, or any synthetic variant of such a molecule.

As used herein, the term “AML” includes any molecule having a biological activity which is substantially similar to that of a defensin, cathelicidin, and/or thrombocidin, includes any molecule which substantially promotes the biological activity of a defensin, cathelicidin, and/or thrombocidin, includes any molecule which is substantially structurally homologous to a defensin, cathelicidin and/or thrombocidin.

The method may be effected using a single regulator of the present invention, or using any combination of multiple regulators of the present invention.

The AMP/AML inhibitor may be: a molecule capable of binding the AMP/AML; an enzyme capable of cleaving the AMP/AML; an siRNA molecule capable of inducing degradation of an mRNA encoding the AMP/AML; a DNAzyme capable of cleaving an mRNA or DNA encoding the AMP/AML; an antisense polynucleotide capable of hybridizing with an mRNA encoding the AMP/AML; a ribozyme capable of cleaving an mRNA encoding the AMP/AML; a non-functional analogue of at least a functional portion of the AMP/AML; a molecule capable of inhibiting activation or ligand binding of the AMP/AML; and a triplex-forming oligonucleotide capable of hybridizing with a DNA encoding the AMP/AML.

Ample guidance for obtaining and utilizing such AMP/AML inhibitors is provided hereinbelow and in the literature of the art (for example, refer to U.S. Patent Application No. 20030044907 which is incorporated herein by reference).

The AMP/AML inhibitor may be any small molecule, AMP/AML dominant negative, or polypeptide that competes with the AMPs for cognate cell receptors without inducing disease. For example, the AMP/AML inhibitor may be a topological analogue of an AMP/AML that has been engineered to remain anti microbial yet lose its chemoattracting ability. Engineering of disulfide bridges to dissect antimicrobial and chemotactic activities of AMPs/AMLs such as human beta-defensin-3 can be performed as previously described (Wu Z. et al., 2003. Proc. Natl. Acad. Sci. U.S.A. 100:8880-5).

The AMP/AML inhibitor may be a synthetic antibody mimic in which multiple peptide loops are attached to a molecular scaffold (described in U.S. Pat. No. 5,770,380).

Such an AMP/AML mimic can be obtained, for example, by molecule imprinting. This technique may be performed by preparing a polymer by cross-linking a monomer around a “template molecule” (the AMP/AML). This template molecule is removed after the polymerization of the monomer and its size, shape and chemical functions are recorded in the polymer. The sites of the removed template molecule are named “imprint sites”. These sites allow the recognition of the template molecule or close structural molecules. Molecularly imprinted polymers can serve as artificial binding mimics as do natural antibodies.

The molecule capable of inhibiting activation or ligand binding of the AMP/AML may advantageously inhibit binding of a receptor expressed on cell, such as a leukocyte, which binds the AMP/AML to inhibit a biological process mediated by binding of the AMP/AML to the receptor. Examples of such AMPs/AMLs and cognate receptors thereof are shown in Table 1.

TABLE 1
AMPs/AMLs and cognate cell receptors, and diseases associated with interaction therebetween

| AMP/AML | Receptor | Receptor-expressing cells | Disease |
|---|---|---|---|
| LL-37 | EGFR, FPRL1 | Monocyte, dendritic cell, T | Psoriasis, rheumatoid arthritis |
| | | cell, neutrophils, | (RA), atopic dermatitis, contact |
| | | eosinophils, leukocytes, | dermatitis, chronic hepatitis, |
| | | epithelial cell, endothelial | inflammatory bowel disease |
| | | cells | (IBD), allergy, B cell |
| | | | malignancies, hepatocellular |
| | | | carcinoma, pancreatic |
| | | | adenocarcinoma and others |
| beta-defensin-2 | Toll 1-like receptor-4 | Dendritic cells | |
| beta-defensin-2 | Toll-like receptor-2 | | |
| beta defensin-1 | CC-chemokine | Hematopoietic cells, | Psoriasis, RA, atopic dermatitis |
| beta defensin-2 | receptor-6 (CCR6) | dendritic cells, | contact dermatitis, chronic |
| | | | hepatitis, IBD, allergy, B cell |
| | | | malignancies, hepatocellular |
| | | | carcinoma, pancreatic |
| | | | adenocarcinoma and more |
| defensin-5 | | Intestinal mucosa | Crohn's disease |
| adrenomedullin | L1 and calcitonin | gastric epithelial cells | IBD, allergy, hepatocellular |
| | receptor-like receptor | | carcinoma, and more |
| | (CRLR) | | |

Further examples of receptors of AMPs/AMLs such as chemokines, the cells in which such receptors are expressed, and the diseases in which the interaction between such AMPs/AMLs and such receptors are involved are provided in D'Ambrosio et al., 2003. J. Immunol. Methods 273 3-13.

The activity of LL-37 (Weiner, D J. et al., 2003. Am. J. Respir. Cell Mol. Biol. 28:738-745), defensin-3, lactoferrin and IL-8 (Perks, B. et al., 2000. Am. J. Respir. Crit Care Med. 162:1767-1772) is inhibited by F-actin, therefore the AMP/AML inhibitor may be F-actin. F-actin forms bundles in the presence of the polycationic interleulin IL-8, therefore F-actin is an inhibitor of downstream elements of the ligand-receptor connectivity of both LL-37 and interleukin IL-8. LL-37 and defensin-3 are inhibited by gelsolin, therefore the AMP/AML inhibitor may be gelsolin. Serpins and their analogs or fragments are inactivators of AMP by formation of complexes with AMP (Panyutich, A V. et al., 1995. Am. J. Respir. Cell Mol. Biol. 12:351-357; alpha-1 antichymotrypsin, the antimicrobial proteins alpha PI, SLPI and elafin are serpins that form complexes with other AMPs) thereby reducing specific types of inflammation (Hiemstra, P S, 2002. Biochem. Soc. Trans. 30:116-120), therefore the AMP/AML inhibitor may be serpins and their analogs or fragments. The AMP/AML inhibitor may be SIC, a secreted protein of streptococcus pyogenes that inactivates antibacterial peptides.

Preferably, the molecule capable of binding the AMP/AML is an antibody or an antibody fragment.

Alternately, the molecule capable of binding the AMP/AML may be any of various type of molecule, including non-immunoglobulin peptides and polypeptides,

Preferably, the antibody fragment is selected from the group consisting of a single-chain Fv, an Fab, an Fab′, and an F(ab′)2.

As used herein, the term “antibody” refers to a substantially intact antibody molecule.

As used herein, the phrase “antibody fragment” refers to a functional fragment of an antibody that is capable of binding to an AMP/AML.

Suitable antibody fragments for practicing the present invention include a complementarity-determining region (CDR) of an immunoglobulin light chain (referred to herein as “light chain”), a CDR of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv, an Fab, an Fab′, and an F(ab′)₂.

Functional antibody fragments comprising whole or essentially whole variable regions of both light and heavy chains are defined as follows:

(i) Fv, defined as a genetically engineered fragment consisting of the variable region of the light chain and the variable region of the heavy chain expressed as two chains;

(ii) single chain Fv (“scFv”), a genetically engineered single chain molecule including the variable region of the light chain and the variable region of the heavy chain, linked by a suitable polypeptide linker.

(iii) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and C_{H}1 domains thereof;

(iv) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule); and

(v) F(ab′)₂, a fragment of an antibody molecule containing a monovalent antigen-binding portion of an antibody molecule which can be obtained by treating whole antibody with the enzyme pepsin (i.e., a dimer of Fab′ fragments held together by two disulfide bonds).

Methods of generating antibodies (i.e., monoclonal and polyclonal) are well known in the art. Antibodies may be generated via any one of several methods known in the art, which methods can employ induction of in-vivo production of antibody molecules, screening of immunoglobulin libraries (Orlandi D. R. et al., 1989. Proc. Natl. Acad. Sci. U.S.A. 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U.S.A. 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumin (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments can be obtained using methods well known in the art. [(see, for example, Harlow and Lane, “Antibodies: A Laboratory Manual”, Cold Spring Harbor Laboratory, New York, (1988)]. For example, antibody fragments according to the present invention can be prepared by proteolytic hydrolysis of the antibody or by expression in E. coli or mammalian cells (e.g., Chinese hamster ovary cell culture or other protein expression systems) of DNA encoding the fragment.

Alternatively, antibody fragments can be obtained by pepsin or papain digestion of whole antibodies by conventional methods. As described hereinabove, an (Fab′)₂ antibody fragments can be produced by enzymatic cleavage of antibodies with pepsin to provide a 5S fragment. This fragment can be further cleaved using a thiol reducing agent, and optionally a blocking group for the sulfhydryl groups resulting from cleavage of disulfide linkages to produce 3.5S Fab′ monovalent fragments. Alternatively, enzymatic cleavage using pepsin produces two monovalent Fab′ fragments and an Fc fragment directly. Ample guidance for practicing such methods is provided in the literature of the art (for example, refer to: Goldenberg, U.S. Pat. Nos. 4,036,945 and 4,331,647; Poiter, R R., 1959. Biochem. J. 73:119-126). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, an Fv is composed of paired heavy chain variable and light chain variable domains. This association may be noncovalent (see, for example, Inbar et al., 1972. Proc. Natl. Acad. Sci. USA. 69:2659-62). Alternatively, as described hereinabove the variable domains can be linked to generate a single chain Fv by an intermolecular disulfide bond, or alternately, such chains may be cross-linked by chemicals such as glutaraldehyde.

Preferably, the Fv is a single chain Fv.

Single chain Fv's are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains. Ample guidance for producing single chain Fv's is provided in the literature of the art (for example, refer to: Whitlow and Filpula, 1991. Methods 2:97-105; Bird et al., 1988. Science 242:423-426; Pack et al., 1993. Bio/Technology 11:1271-77; and Ladner et al., U.S. Pat. No. 4,946,778).

Isolated complementarity determining region peptides can be obtained by constructing genes encoding the complementarity determining region of an antibody of interest. Such genes may be prepared, for example, by RT-PCR of mRNA of an antibody-producing cell. Ample guidance for practicing such methods is provided in the literature of the art (for example, refer to Larrick and Fry, 1991. Methods 2:106-10).

It will be appreciated that for human therapy or diagnostics, humanized antibodies are preferably used. Humanized forms of non human (e.g., murine) antibodies are genetically engineered chimeric antibodies or antibody fragments having-preferably minimal-portions derived from non human antibodies. Humanized antibodies include antibodies in which complementary determining regions of a human antibody (recipient antibody) are replaced by residues from a complementarity determining region of a non human species (donor antibody) such as mouse, rat or rabbit having the desired functionality. In some instances, Fv framework residues of the human antibody are replaced by corresponding non human residues. Humanized antibodies may also comprise residues which are found neither in the recipient antibody nor in the imported complementarity determining region or framework sequences. In general, the humanized antibody will comprise substantially all of at least one, and typically two, variable domains, in which all or substantially all of the complementarity determining regions correspond to those of a non human antibody and all, or substantially all, of the framework regions correspond to those of a relevant human consensus sequence. Humanized antibodies optimally also include at least a portion of an antibody constant region, such as an Fc region, typically derived from a human antibody (see, for example, Jones et al., 1986. Nature 321:522-525; Riechmann et al., 1988. Nature 332:323-329; and Presta, 1992. Curr. Op. Struct. Biol. 2:593-596).

Methods for humanizing non human antibodies are well known in the art. Generally, a humanized antibody has one or more amino acid residues introduced into it from a source which is non human. These non human amino acid residues are often referred to as imported residues which are typically taken from an imported variable domain. Humanization can be essentially performed as described (see, for example: Jones et al., 1986. Nature 321:522-525; Riechmann et al., 1988. Nature 332:323-327; Verhoeyen et al., 1988. Science 239:1534-1536; U.S. Pat. No. 4,816,567) by substituting human complementarity determining regions with corresponding rodent complementarity determining regions. Accordingly, such humanized antibodies are chimeric antibodies, wherein substantially less than an intact human variable domain has been substituted by the corresponding sequence from a non human species. In practice, humanized antibodies may be typically human antibodies in which some complementarity determining region residues and possibly some framework residues are substituted by residues from analogous sites in rodent antibodies.

Human antibodies can also be produced using various techniques known in the art, including phage display libraries [see, for example, Hoogenboom and Winter, 1991. J. Mol. Biol. 227:381; Marks et al., 1991. J. Mol. Biol. 222:581; Cole et al., “Monoclonal Antibodies and Cancer Therapy”, Alan R. Liss, pp. 77 (1985); Boerner et al., 1991. J. Immunol. 147:86-95). Humanized antibodies can also be made by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807, 5,545,806, 5,569,825, 5,625,126, 5,633,425, and 5,661,016; Marks et al., 1992. Biotechnology 10:779-783; Lonberg et al., 1994. Nature 368:856-859; Morrison, 1994. Nature 368:812-13; Fishwild et al., 1996. Nature Biotechnology 14:845-51; Neuberger, 1996. Nature Biotechnology 14:826; Lonberg and Huszar, 1995. Intern. Rev. Immunol. 13:65-93; Kellermann, S A. et al., 2002. Curr. Op. Biotechnol. 13:593-597).

Once antibodies are obtained, they may be tested for activity, for example via ELISA.

Suitable antibodies may in many cases be purchased ready for use from commercial suppliers, such as Pharmingen, Dako, Becton-Dickinson, Sigma-Aldrich, and the like. Algae can be used to industrially mass-produce antibodies (Proc Natl Acad Sci USA. 2003, 100:438-42).

As described hereinabove, the AMP/AML inhibitor may be a small interfering RNA (siRNA) molecule. RNA interference is a two step process. the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl Chem Biochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the AMP/AML mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl Chem Biochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

As used herein the term “about” refers to plus or minus 10%.

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

As described hereinabove, the AMP/AML inhibitor may be a DNAzyme molecule capable of specifically cleaving an mRNA transcript or DNA sequence of the AMP/AML. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al.

As described hereinabove, the AMP/AML inhibitor may be an antisense polynucleotide capable of specifically hybridizing with an mRNA transcript encoding the AMP/AML.

Design of antisense molecules which can be used to efficiently decrease levels/activity of an AMP/AML must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

As described hereinabove, the AMP/AML inhibitor may be a ribozyme molecule capable of specifically cleaving an mRNA transcript encoding the AMP/AML. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

As described hereinabove, the AMP/AML inhibitor may be a triplex forming oligonucleotides (TFOs). TFOs can be used for regulating the expression of an AMP/AML gene in cells. Recent studies have shown that TFOs can be designed which can recognize and bind to polypurine/polypyrimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence: oligo, 3-A G G T; duplex, 5′-A G C T; and duplex, 3′-T C G A.

However, it has been shown that the A-AT and G-GC triplets have the greatest triple helical stability (Reither and Jeltsch, BMC Biochem, 2002, Sept. 12, Epub). The same authors have demonstrated that TFOs designed according to the A-AT and G-GC rule do not form non-specific triplexes, indicating that the triplex formation is indeed sequence specific.

Thus for any given sequence in the AMP/AML gene a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

Techniques for administering such molecules to a cell or cellular structure are routinely practiced by the ordinarily skilled artisan, and ample guidance is provided in the literature of the art for such administration (refer, for example, to the references relevant to such molecules cited hereinabove and to U.S. Patent Application No. 20030044907 which is incorporated herein by reference).

As described hereinabove, the method of regulating the biological process of the present invention comprises the step of exposing the cell/tissue to the regulator.

Exposing the cell/tissue to the regulator may be effected in various ways depending on the application and purpose. In cases where the cell/tissue form part of a human or an animal subject, exposing the cell/tissue to the regulator is preferably effected by providing the regulator to the subject.

Administering the regulator to a subject may be effected via any suitable route facilitating exposure of the cell/tissue with the regulator, including a route selected from the group consisting of the topical, intranasal, transdermal, intradermal, oral, buccal, parenteral, rectal and inhalation route.

Preferably, subcutaneous and/or local injection of the regulator in saline solution is used for treating a disease such as arthritis.

Preferably, topical application of the regulator in lipid or saline solution, or in a cream on the skin is used for treating a cutaneous disease such as a psoriasis legion.

Preferably, for treating respiratory diseases such as cystic fibrosis and asthma, the regulator dissolved in a solution and administered using an inhaler.

Alternately, the cells may be exposed to regulator by expressing the regulator in the human or animal. In cases where the cell/tissue is a cultured cell/tissue, exposing the regulator to the cell/tissue is preferably effected by providing the regulator to the cell/tissue in-vitro using standard tissue culture methods. Preferably, providing the regulator to the cell/tissue in-vitro is effected as described in the Examples section which follows.

The regulator can be expressed in a subject by directly administering to the subject a nucleic acid construct configured so as to suitably express the regulator in-vivo. Alternatively, a nucleic acid construct for expressing the regulator may be introduced into a suitable cell ex-vivo via an appropriate gene delivery vehicle/method (transfection, transduction, homologous recombination, etc.), and using a suitable genetic expression system as needed. The modified cells may be expanded in culture and administered to the subject where they will produce the regulator in-vivo. To enable cellular expression of the regulator, a nucleic acid construct which encodes the regulator preferably includes at least one cis acting regulatory element, most preferably a promoter which is active in the specific cell population transformed. The nucleic acid construct can further include an enhancer, which can be adjacent or distant to the promoter sequence and can function in up regulating the transcription therefrom.

Suitable in vivo nucleic acid transfer techniques include transfection with viral or non-viral constructs, such as adenovirus, lentivirus, Herpes simplex I virus, or adeno-associated virus (AAV) and lipid-based systems, polylysine based systems and dendrimers. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. The construct may include a signal that directs polyadenylation, as well as one or more restriction sites and a translation termination sequence. By way of example, such a constructs will typically include a 5′ LTR, a tRNA binding site, a packaging signal, an origin of second-strand DNA synthesis, and a 3′ LTR or a portion thereof.

The various aspects of the present invention may be practiced by using, and/or by decreasing the activity/level, of any of various types of AMPs/AMLs, depending on the application and purpose.

Preferably, the AMP/AML is a cationic and/or hydrophobic peptide.

As used herein, the term “peptide” (with the exception of the term in the context of the phrases “antimicrobial peptide” or “antimicrobial-like peptide”, refers to a polypeptide which is composed of less than 51 amino acid residue.

Preferably, the AMP/AML is a defensin or a cathelicidin.

Preferably, the defensin is a beta-defensin, most preferably beta-defensin-1 or beta-defensin-2.

Preferably, the cathelicidin is LL-37.

Preferably, the AMP/AML is of human origin. Alternately, it may be of non-human origin, in which case it is preferably of mammalian origin.

Numerous examples of AMPs/AMLs which may be used, and/or whose activity/levels may be decreased, for practicing the various aspects of the present invention are described in further detail hereinbelow.

The method may be practiced so as to regulate the biological process in any of various cells/tissues of the present invention.

Preferably, the method is used to regulate the biological process in an epithelial cell/tissue, an endothelial cell/tissue, a gastrointestinal tissue, and/or a tumor cell/tissue.

The cell/tissue is preferably an epithelial, skin, endothelial, gastrointestinal, and/or tumor cell/tissue.

The method may be used to regulate the biological process in any of various types of skin cells/tissues.

Preferably, the skin cell/tissue is a keratinocytic cell/tissue.

Preferably, the gastrointestinal cell/tissue is a gastrointestinal epithelial cell/tissue.

Preferably, the tumor cell/tissue is a malignant cell/tissue. Alternately, the tumor cell/tissue may be a benign tumor cell/tissue.

Preferably, the tumor cell/tissue is a metastatic tumor cell/tissue.

The method may be used to regulate the biological process in a tumor cell/tissue which is of any of various cell/tissue types.

Preferably, the malignant cell/tissue is a skin cell/tissue.

The method may be effected by exposing the cell/tissue to the regulator at any of various concentrations, depending on the application and purpose.

Preferably, when using an AMP/AML inhibitor of the present invention for regulating the biological process, exposing the cell/tissue to the AMP/AML inhibitor is effected by exposing the cell/tissue to the AMP/AML inhibitor at a concentration selected from a range of about 50 nanograms per milliliter to about one milligram per milliliter.

Exposing the cell/tissue to the AMP/AML inhibitor may advantageously be effected, depending on the application and purpose, by exposing the cell/tissue to the AMP/AML inhibitor at a concentration selected from a range of about 50 ng/ml to about 100 micrograms/ml, from a range of about 100 micrograms/ml to about 200 micrograms/ml, from a range of about 200 micrograms/ml to about 300 micrograms/ml, from a range of about 300 micrograms/ml to about 400 micrograms/ml, from a range of about 400 micrograms/ml to about 500 micrograms/ml, from a range of about 500 micrograms/ml to about 600 micrograms/ml, from a range of about 600 micrograms/ml to about 700 micrograms/ml, from a range of about 700 micrograms/ml to about 800 micrograms/ml, from a range of about 800 micrograms/ml to about 900 micrograms/ml, from a range of about 900 micrograms/ml to about 1 mg/ml.

Preferably, when using an AMP/AML of the present invention for regulating the biological process, exposing the cell/tissue to the AMP/AML is effected by exposing the cell/tissue to the AMP/AML at a concentration selected from a range of about 2 ng/ml to about 10 micrograms/ml.

Exposing the cell/tissue to the AMP/AML may advantageously be effected, depending on the application and purpose, by exposing the cell/tissue to the AMP/AML inhibitor at a concentration selected from a range of about 2 ng/ml to about 1 microgram/ml, from a range of about 1 microgram/ml to about 2 micrograms/ml, from a range of about 2 micrograms/ml to about 3 micrograms/ml, from a range of about 3 micrograms/ml to about 4 micrograms/ml, from a range of about 4 micrograms/ml to about 5 micrograms/ml, from a range of about 5 micrograms/ml to about 6 micrograms/ml, from a range of about 6 micrograms/ml to about 7 micrograms/ml, from a range of about 7 micrograms/ml to about 8 micrograms/ml, from a range of about 8 micrograms/ml to about 9 micrograms/ml, from a range of about 9 micrograms/ml to about 10 micrograms/ml.

The method can be used to regulate in the cell/tissue a biological process such as growth, differentiation, inflammation, metastasis and/or angiogenesis.

For regulating growth in an epithelial, skin and/or gastrointestinal cell/tissue, the regulator may advantageously be an AMP/AML inhibitor of the present invention and/or an AMP/AML of the present invention.

For inducing growth in an epithelial, skin and/or gastrointestinal cell/tissue, the regulator used is preferably a defensin inhibitor of the present invention and/or a cathelicidin inhibitor of the present invention.

As used herein, the phrase “defensin inhibitor” refers to a compound of the present invention which is capable of decreasing an activity and/or level of a defensin.

As used herein, the phrase “cathelicidin inhibitor” refers to a compound of the present invention which is capable of decreasing an activity and/or level of a cathelicidin.

Preferably, for inducing growth in an epithelial and/or skin cell/tissue the AMP/AML is preferably a defensin. Preferably the defensin is used for such purpose at a concentration selected from a range of about 0.1 microgram/ml to about 10 micrograms/ml, most preferably at a concentration of about 1 microgram/ml.

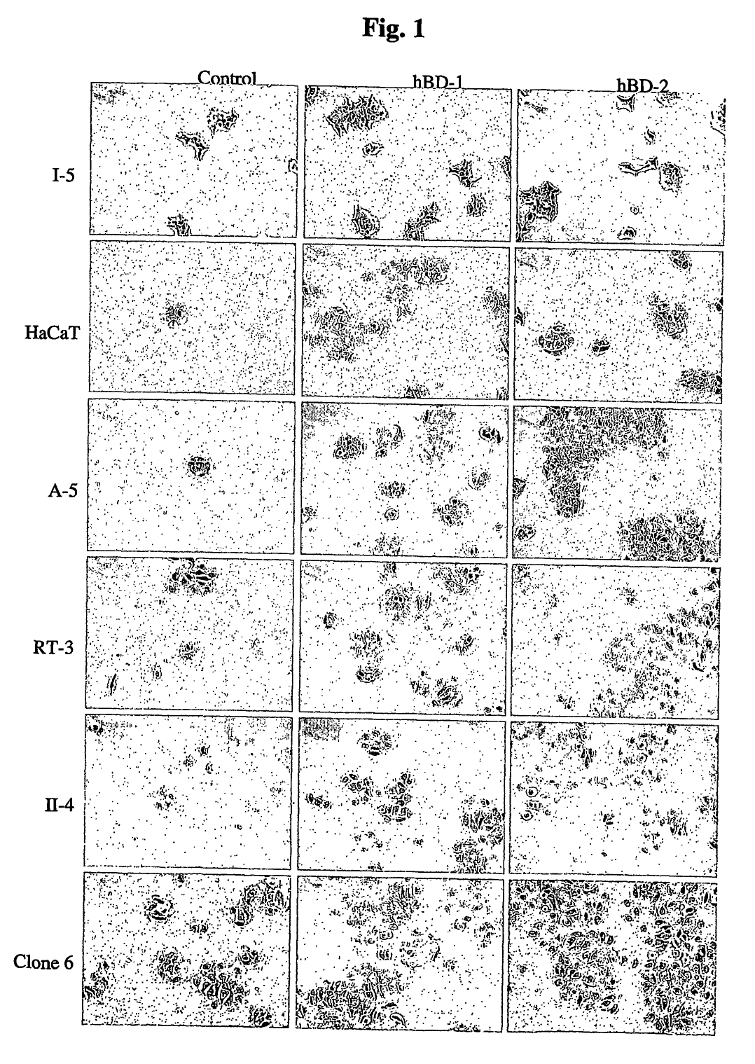

As is shown in Example 1 (FIG. 1) of the Examples section below, beta-defensin-1 or beta-defensin-2 at a concentration of I microgram/ml can be used to induce growth in a human skin cell/tissue.

Preferably, for inducing growth in an epithelial, skin and/or gastrointestinal cell/tissue a defensin inhibitor is used at a concentration selected from a range of about 50 ng/ml to about 50 micrograms/ml. Preferably the defensin inhibitor employed for such purpose is a beta-defensin-2 inhibitor of the present invention.

As used herein, the phrase “beta-defensin-2 inhibitor” refers to a compound of the present invention which is capable of decreasing an activity and/or level of beta-defensin-2.

Preferably, for inducing growth in a skin and/or keratinocytic cell/tissue, the defensin inhibitor is used at a concentration of about 0.1 microgram/ml to about 10 micrograms/ml, most preferably at a concentration of about 1 microgram/ml. As is shown in Example 2 of the Examples section (FIG. 3), anti-beta-defensin-2 antibody at a concentration of 1 microgram/ml can be used to induce growth of primary human skin cells.

Preferably, for inducing growth in a skin and/or keratinocytic cell/tissue, the cathelicidin inhibitor is used at a concentration of about 0.4 microgram/ml to about 40 micrograms/ml, most preferably at a concentration of about 4 micrograms/ml. As is shown in Example 2 of the Examples section (FIG. 3), anti-LL-37 antibody at a concentration of 4 micrograms/ml can be used to induce growth of primary human skin cells.

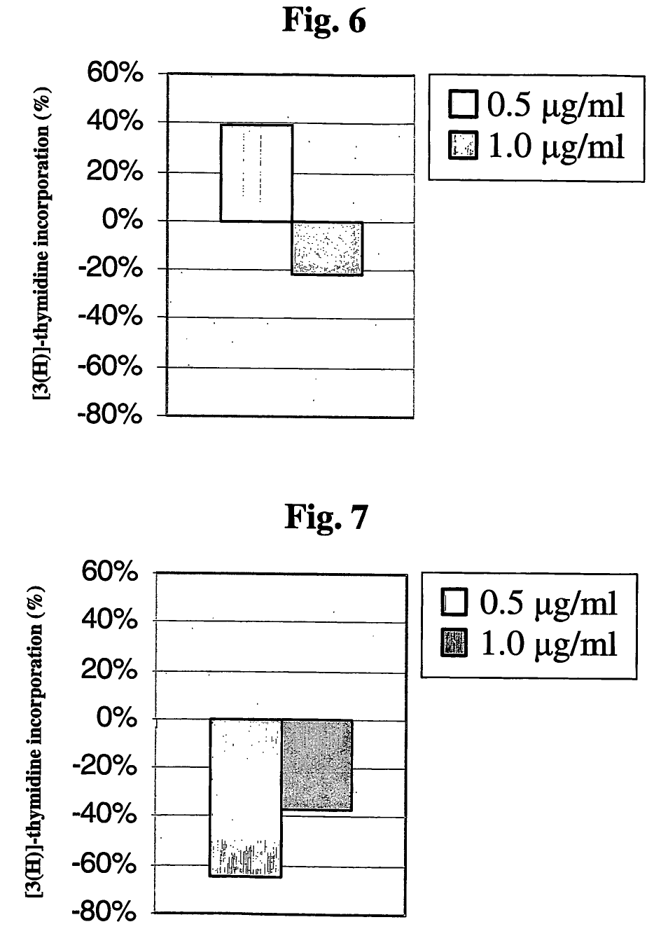

Preferably, for inducing growth in a gastrointestinal cell/tissue, the defensin inhibitor is used at a concentration selected from a range of about 50 ng/ml to about 5 micrograms/ml, most preferably at a concentration of about 0.5 microgram/ml. As is shown in Example 4 of the Examples section (FIG. 6), anti-beta-defensin-2 antibody at a concentration of 0.5 microgram/ml can be used to induce growth of a human gastrointestinal epithelial cell/tissue.

For inhibiting growth in a tumor, epithelial, skin and/or gastrointestinal cell/tissue, the regulator used may advantageously be a defensin inhibitor of the present invention, and/or a cathelicidin inhibitor of the present invention. For such purpose the defensin inhibitor is preferably a beta-defensin-2 inhibitor of the present invention.

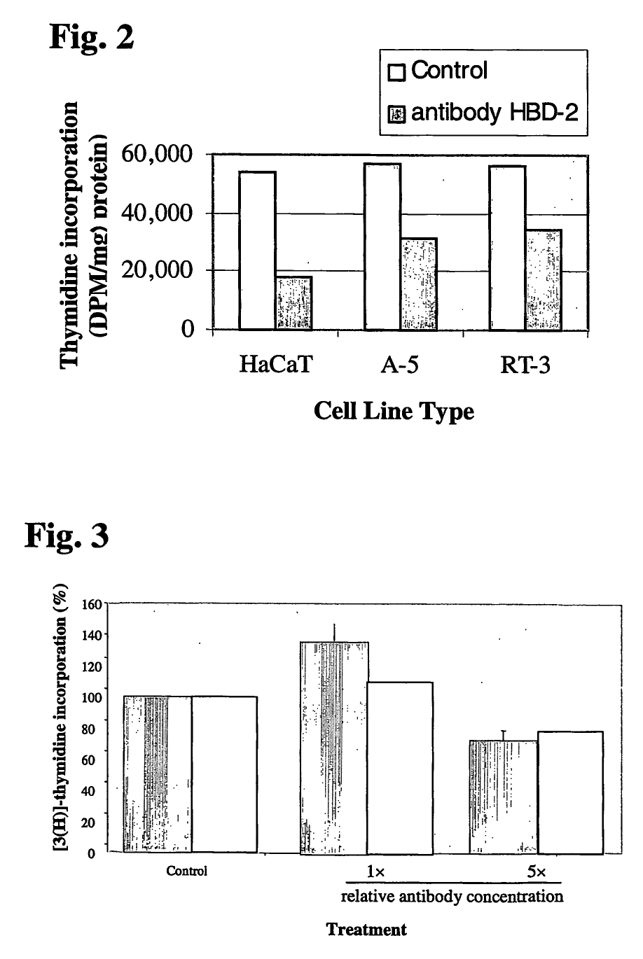

Preferably, for inhibiting growth in a tumor cell/tissue the defensin inhibitor is used at a concentration selected from a range of about 0.1 microgram/ml to about 10 micrograms/ml, more preferably at a concentration of about 1 microgram/ml. As is shown in Example 1 of the Examples section (FIG. 2), anti-beta-defensin-2 antibody at a concentration of 1 microgram/ml can be used to inhibit growth of a malignant skin carcinoma cell/tissue.

Preferably, for inhibiting growth in a skin and/or keratinocytic cell/tissue, the defensin inhibitor is used at a concentration of about 50 ng/ml to about 50 micrograms/ml, most preferably at a concentration of about 5 micrograms/ml. As is shown in Example 2 of the Examples section (FIG. 3), anti-beta-defensin-2 antibody at a concentration of 5 micrograms/ml can be used to inhibit growth of primary human skin cells.

Preferably, for inhibiting growth in a skin and/or keratinocytic cell/tissue, the cathelicidin inhibitor is used at a concentration of about 2 micrograms/ml to about 200 micrograms/ml, most preferably at a concentration of about 20 micrograms/ml. As is shown in Example 2 of the Examples section (FIG. 3), anti-cathelicidin antibody at a concentration of 20 micrograms/ml can be used to inhibit growth of primary human skin cells.

Preferably, for inhibiting growth in a gastrointestinal cell/tissue, the defensin inhibitor is used at a concentration selected from a range of about 0.1 microgram/ml to about 10 micrograms/ml, most preferably at a concentration of about 1 microgram/ml. As is shown in Example 4 of the Examples section (FIG. 6), anti-beta-defensin-2 antibody at a concentration of 1 microgram/ml can be used to inhibit growth of a human gastrointestinal epithelial cell/tissue.

For inhibiting angiogenesis/endothelial cell/tissue growth, the regulator used is preferably a defensin inhibitor. Preferably, for such purpose, the defensin inhibitor used is a beta-defensin-2 inhibitor of the present invention. Preferably, the defensin inhibitor is used at a concentration selected from a range of about 50 nanograms/ml to about 10 micrograms/ml, more preferably from a range of about 50 ng/ml to about 5 micrograms/ml and most preferably is used at a concentration of about 0.5 microgram/ml. As is shown in Example 5 of the Examples section (FIG. 7), anti-beta-defensin-2 antibody at a concentration of 0.5 or 1 microgram/ml (especially 0.5 microgram/ml) can be used to inhibit angiogenesis/human endothelial cell/tissue growth.

For inhibiting metastasis in a tumor cell/tissue, the regulator used is preferably a defensin inhibitor of the present invention. Preferably, for such purpose, the defensin inhibitor is a beta-defensin-2 inhibitor of the present invention. Preferably, the defensin inhibitor is used for such a purpose at a concentration selected from a range of about 0.1 microgram/ml to about 10 micrograms/ml, most preferably at a concentration of about 1 microgram/ml.

As is described in Example 1 of the Examples section below anti-beta-defensin-2 antibody at a concentration of 1 microgram/ml can be used to inhibit substrate detachment of human malignant skin tumor cells/tissue.

For correcting dysregulated balance of proliferation/differentiation in an epithelial and/or skin cell/tissue, the regulator is preferably a defensin inhibitor. Preferably, the defensin inhibitor used for such a purpose is a beta-defensin-2 inhibitor of the present invention. Preferably, the beta-defensin-2 inhibitor is used for such a purpose at a concentration selected from a range of about 0.1 microgram/ml to about 1 mg/ml, most preferably at a concentration of about 1 microgram/ml or 100 micrograms/ml.

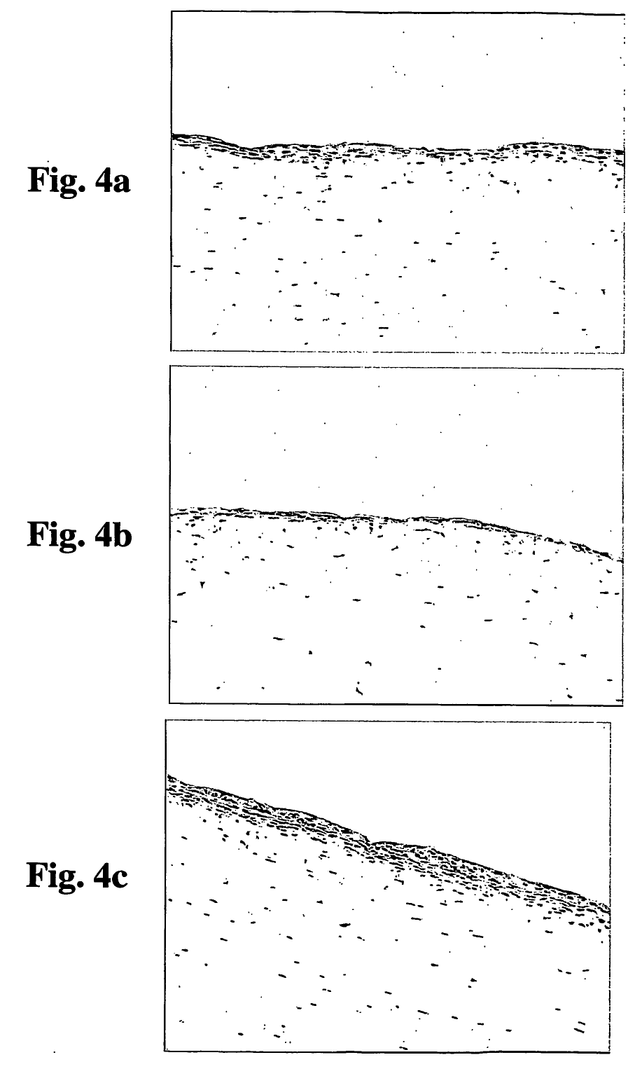

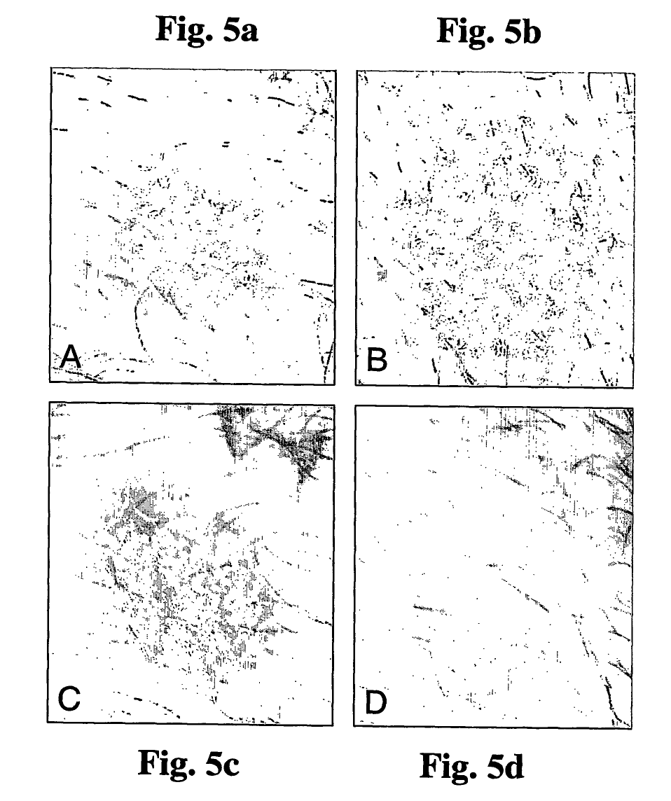

As is shown in Example 3 (FIGS. 4a-c) of the Examples section below anti-beta-defensin-2 antibody at a concentration of 1 microgram/ml can be used to correct proliferation/differentiation imbalance in a highly realistic three-dimensional organotypic in-vitro human skin model. As is shown in Example 3 of the Examples section which follows (FIGS. 5a-d), anti-beta-defensin-2 antibody at a concentration of 100 micrograms/ml can be used to inhibit flaking in a human psoriasis lesion, indicating correction of skin cell/tissue proliferation/differentiation imbalance.

For inhibiting inflammation in a cell/tissue, the regulator used is preferably a defensin inhibitor of the present invention. Preferably, for such purpose, the defensin inhibitor is a beta-defensin-2 inhibitor of the present invention. Preferably, the defensin inhibitor is used for such a purpose at a concentration selected from a range of about 50 ng/ml to about 1 mg/ml, most preferably at a concentration of about 0.5 microgram/ml or about 100 micrograms/ml.

As is shown in Example 3 (FIGS. 5a-d) of the Examples section below anti-beta-defensin-2 antibody at a concentration of 1 microgram/ml can be used to inhibit an autoimmune inflammation in a human tissue. As is further shown in Example 5 of the Examples section which follows (FIG. 7), anti-beta-defensin-2 antibody at a concentration of 0.5 microgram/ml can be used to inhibit human endothelial cell/tissue growth, indicating a capacity for inhibition of angiogenesis by the regulator. It will be appreciated that by virtue of enabling inhibition of an inflammation in a human tissue, and by virtue of enabling inhibition of angiogenesis, that the presently described method enables potent inhibition of inflammation.

As described hereinabove, the present invention can be used for regulating biological processes such as growth, differentiation, inflammation, metastasis and angiogenesis. It will be appreciated that such biological processes are associated with the pathogenesis of numerous diseases, and that regulation of such biological processes according to the teachings of the present invention can be used for treating such diseases.

Thus, according to one aspect of the present invention there is provided a method of treating a disease in a subject in need thereof. The method is effected by providing to the subject a therapeutically effective amount of a compound which is capable of decreasing an activity and/or level of an AMP and/or AMP-like molecule (AML).

As used herein, the term “disease” refers to any medical disease, disorder, condition, or syndrome, or to any undesired and/or abnormal physiological morphological, cosmetic and/or physical state and/or condition.

Herein, the term “treating” includes abrogating, substantially inhibiting, slowing or reversing the progression of a disease, substantially ameliorating clinical symptoms of a disease or substantially preventing the appearance of clinical symptoms of a disease.

The method can be used for treating any of various diseases.

In particular, the method can be used for treating any of various diseases which are associated with: (i) a tumor; (ii) inflammation; (iii) an epithelial wound; (iv) dysregulation of growth/differentiation of a cell/tissue; (v) dysregulation of growth/differentiation balance of a cell/tissue; and (vi) diseases associated with angiogenesis.

Examples of such diseases, and others, which are amenable to treatment via the present invention are listed hereinbelow.

One of ordinary skill in the art, such as a physician, most preferably a physician specialized in the disease, will possess the necessary expertise for treating a disease according to the teachings of the present invention.

As used herein, the phrase “subject in need thereof” refers to a subject having the disease.

Preferably, the subject is a mammal, most preferably a human.

By virtue of demonstrably enabling induction of growth in an epithelial, skin and/or gastrointestinal cell/tissue, the method described above for inducing such growth is particularly suitable for treating any of various diseases in which growth of such tissue will be therapeutic. Such diseases particularly include diseases which are associated with epithelial, skin and/or gastrointestinal wounds.

By virtue of demonstrably enabling growth inhibition of a malignant, epithelial, skin and/or gastrointestinal cell/tissue, the method described above for inhibiting such growth is particularly suitable for treating any of various diseases associated with dysregulated/excessive growth, malignant, epithelial, skin and/or gastrointestinal cell/tissue. Such diseases particularly include tumors in general, gastrointestinal tumors, and malignant skin carcinomas in particular.

By virtue of demonstrably enabling growth inhibition of an endothelial cell/tissue, the method described above for inhibiting such growth is particularly suitable for treating any of various diseases associated with dysregulated/excessive growth of an endothelial cell/tissue, and hence can be used for treating any of various diseases associated with angiogenesis. Such diseases notably include solid tumors, endothelial tumors, and inflammatory diseases including autoimmune diseases such as psoriasis.

By virtue of demonstrably enabling correction of dysregulation of growth/differentiation balance in epithelial and/or skin tissue in an in-vitro three-dimensional organotypic skin model, and in-vivo in an inflammatory lesion associated with such dysregulated balance, the method described above for correcting such balance is particularly suitable for treating any of various diseases associated with such dysregulated balance. Such diseases notably include psoriasis and dandruff.

By virtue of demonstrably enabling inhibition of an autoimmune inflammation in a human tissue, the method described above for inhibiting such inflammation is particularly suitable for treating any of various diseases associated with such inflammation. Such diseases notably include autoimmune diseases, such as psoriasis and gastrointestinal autoimmune diseases.

By virtue of demonstrably enabling inhibition of substrate detachment in a human tumor and/or skin cell/tissue, the method described above for inhibiting such detachment is particularly suitable for treating any of various diseases associated with such detachment. Such diseases notably include metastatic tumors, such as metastatic carcinomas, in particular metastatic malignant skin carcinoma.

For treating the disease, the regulator may be administered via any of various suitable regimens.

Preferably, administering the regulator to the subject is effected by administering to the subject a plurality of doses of the AMP/AML inhibitor which is selected from a range of about 2 doses to about 30 doses, wherein each inter dose interval of the plurality of doses is selected from a range of about 2.4 hours to about 30 days.

Depending on the application and purpose, the plurality of doses may advantageously be selected from a range of about 2 to about 5 doses, from a range of about 5 to about 10 doses, from a range of about 10 to about 15 doses, from a range of about 15 to about 20 doses, from a range of about 20 to about 25 doses, from a range of about 25 to about 30 doses, or from a range of about 30 to about 35 doses.

Preferably, administering the regulator to the subject is effected by administering to the subject 3 doses of the AMP/AML inhibitor.

Depending on the application and purpose, each inter dose interval of the plurality of doses may advantageously be selected from a range of about 2.4 hours to about 3 days, from a range of about 3 days to about 6 days, from a range of about 6 days to about 9 days, from a range of about 9 days to about 12 days, from a range of about 12 days to about 15 days, from a range of about 15 days to about 18 days, from a range of about 18 days to about 21 days, from a range of about 21 days to about 24 days, from a range of about 24 days to about 27 days, or from a range of about 27 days to about 30 days.

Preferably, the inter dose interval of the plurality of doses is about 1 day.

As is described in Example 3 of the Examples section which follows, administering 3 doses of a regulator of the present invention to the subject with an inter dose interval of about 1 day can be used for effectively treating a disease such as psoriasis in a human subject.

Disease treatment may be effected via polytherapy by administration of the regulator in conjunction with peptide inhibitors such as protease inhibitors, the serpin serine proteinase inhibitory components (alpha-1 PI) and alpha-1 antichymotrypsin (Panyutich, A V. et al., 1995. Am. J. Respir. Cell Mol. Biol. 12:351-357), BAPTA-AM (an intracellular Ca(2+) chelating agent), pertussis toxin and U-73122 (a phospholipase C inhibitor; Niyonsaba, F. et al., 2001. Eur. J. Immunol. 31:1066-1075), T-cell targeted therapies, monoclonal antibody against chemokine tumor necrosis factor and cytokine targeted therapies, fibroblast growth factor inhibitors. For example, topical treatments may advantageously include cell proliferation regulators such as retinoid—vitamin A—analog which modulates or changes the cellular differentiation of the epidermis. Such polytherapy may be effected using anti-inflammatory drugs/treatments as a precautionary measure against relapse of psoriasis or other auto-immune disease. Such drugs/treatments include tazarotene, methotrexate, acitretin, bexarotene, ploralem, etretinate, corticosteroid creams and ointments, synthetic vitamin D3, IL-10, IL-4 and IL-1RA (receptor antagonist).

To enable treatment of the disease, the regulator is preferably included as an active ingredient in a pharmaceutical composition which includes a suitable carrier and which is suitably packaged and labeled for treatment of the disease.

The regulator according to the present invention can be administered to a subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of active ingredients to an organism.

Herein the term “active ingredients” refers to the regulator of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered active ingredients. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transinucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the active ingredients and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions. Cream solutions can include any lipids or organic alcohols or chemicals including for example benzyl alcohol, macrogol, hexylene glycol, carbomer, ascorbic acid, butyl hydroxyainisole, butyl hydroxytoluene, disodium edentate, water, trometamol, poxoamer.

Alternatively, the active ingredients may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (regulator of the present invention) effective to prevent, alleviate or ameliorate symptoms of a disorder (e.g., psoriasis or a carcinoma) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredients which are sufficient to achieve a desired therapeutic effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Thus, the present invention provides an article of manufacture which comprises packaging material identified for treatment of the disease, and a pharmaceutical composition which includes a pharmaceutically acceptable carrier and, as an active ingredient, the regulator.

Preferably, the pharmaceutical composition is formulated as a solution, suspension, emulsion or gel.

Preferably, the pharmaceutically acceptable carrier is selected so as to enable administration of the pharmaceutical composition via a route selected from the group consisting of the topical, intranasal, transdermal, intradermal, oral, buccal, parenteral, rectal and inhalation route.

Preferably, the pharmaceutical composition is composed so as to enable exposure of an affected cell/tissue of the subject having the disease, to the regulator at a suitable concentration, as described hereinabove, for treating the disease.

Preferably, the pharmaceutical composition is further identified for administration to the subject according to a suitable regimen, as described hereinabove.

Thus, the present invention provides a method of identifying a compound capable of regulating the biological process in a cell/tissue. The method is effected in a first step by exposing the cell/tissue to a test compound which is: a compound capable of decreasing an activity and/or level of an antimicrobial peptide (AMP) and/or AMP-like molecule (AML); and/or which is the AMP and/or AML. In a second step, the method is effected by evaluating a capacity of the test compound to regulate the biological process in the cell and/or tissue.

It will be appreciated that the method of identifying the compound can be used for screening a plurality of compounds so as to identify a compound having a desired capacity for regulating a biological process.

The method is preferably used to identify a compound capable of regulating a biological process as described hereinabove with respect to the method of the present invention of regulating a biological process.

Preferably, the test compound is a regulator as described hereinabove with respect to the method of the present invention of regulating a biological process.

The method is preferably used to identify a compound capable of regulating the biological process in the cell/tissue as described hereinabove with respect to the method of the present invention of regulating a biological process, and as described in the Examples section which follows. As is described hereinabove with respect to the method of regulating the biological process, and in the Examples section which follows, the method is preferably employed for identifying a compound which is capable of: inducing growth in an epithelial, skin, keratinocytic and/or gastrointestinal cell/tissue; inhibiting growth in a tumor, epithelial, skin, keratinocytic and/or gastrointestinal cell/tissue; inhibiting angiogenesis/endothelial cell/tissue growth; inhibiting metastasis in a tumor cell/tissue; correcting dysregulated balance of proliferation/differentiation in an epithelial, keratinocytic and/or skin cell/tissue; and/or inhibiting inflammation in an epithelial, keratinocytic an/or skin cell/tissue.

The identification method may advantageously be performed using high-throughput methodology. Ample guidance for practicing relevant high-throughput methods is provided in the literature of the art (refer, for example, to U.S. Patent Application No. 20030044907).

The test compound may be exposed to the cell/tissue in any of various ways. Preferably, the test compound is exposed to the cell/tissue in-vitro as described in the Examples section which follows. Alternately, the test compound may be exposed to the cell/tissue by exposing the test compound to a cultured cell/tissue.

Preferably, the cell which produces the test compound is a B-cell hybridoma. Alternately, the cell which produces the test compound may be of any of various types, depending on the application and purpose.

It will be appreciated that a B-cell hybridoma is an antibody producing cell, and hence that exposing the cell/tissue to a B-cell hybridoma can be used for identifying a B-cell hybridoma which expresses an antibody which is capable of regulating the biological process.

Exposing the cell/tissue to the test compound may be effected by providing the test compound to a subject which includes the cell/tissue (in-vivo model). Preferably providing the test compound to the test subject is effected as described hereinabove with respect to providing the regulator to a subject.

The identification method may be effected by exposing the test compound to: psoriasis lesions and lesions of any of various diseases associated with epithelial wounds included in the present invention; a psoriatic lesion in a psoriasis animal model; or a psoriatic lesion in a human having psoriasis; a human biopsy of a normal or pathological involved lesion maintained in an organotypic culture containing plasma and lymphocytes of patients suffering from the disease having and not having polymorphism on AMPs or their genes and promoters; and/or to a cell/tissue of a disease in which the disease inductive isoforms are ApoE4 and the non inductive isoform is ApoE3.

The identification method may be effected by exposing the test compound to a human psoriatic lesion biopsy grafted onto an animal (xenograft model), whereby the biopsy is taken with informed consent. The biopsy may be transplanted onto an immunodeficient mouse (for example, NIHS-bg-nu-xid or BNX). For establishing such a xenograft model, PBMCs may be isolated from the blood obtained from the biopsy donor and activated (for example, using a superantigen), and the animals injected with the activated PBMCs. Ample guidance for practicing the identification method using such animal models is provided in Examples 6-8 of the Examples section below and in the literature of the art (refer, for example, to U.S. Patent Application No. 20030044907).

The most affected tissue in psoriasis, in addition to the activated immune system, is skin. The main cells composing skin are epidermal keratinocytes and dermal fibroblasts. Other cells include endothelial cells, melanocytes, hair follicle cells, sweat gland cells, and immune system cells. Such cells may advantageously be used to practice the identification method.

Evaluation of the regulation of the biological processes encompassed by the identification method may be effected using any of various suitable methods known to the ordinarily skilled artisan. Preferably, such evaluation is performed, where relevant, as described in the Examples section which follows.

Evaluating regulation of the biological process may be effected using quantitative evaluation of epidermal thickness when using an in-vivo model, cell count or histological evaluation.

Preferably, data obtained from the evaluation is processed using statistical analysis and ANOVA for maximum informativity.

According to one embodiment, the identification method may involve exposing the test compound to cultured microbes/bacteria and evaluating regulation of the biological process is effected by measuring survival of the microbes/bacteria. This may be effected by a colony-forming unit assay performed with Staphylococcus aureus (isolated from clinical sample), GAS (NZ131), and enteroinvasive Escherichia coli 029 as described (Porter et al, 1997). Before analysis, the concentration of the bacteria in culture will be determined by plating different bacterial dilutions. The protocol may be performed as follows. Cells are washed twice with 10 mM sodium phosphate buffer (20 mM NaH₂PO₄.H₂O, 20 mM Na₂HPO₄.7H₂O) and diluted to a concentration of 2,000,000 cells per milliliter (S. aureus, GAS) or 200,000 cells per milliliter (E. coli) in phosphate buffer. S. aureus and E. coli are incubated for 4 hours at 37 degrees centigrade with various concentrations of an AMP/AML in the presence of various concentrations of the test compound to be examined, in 50 microliters of buffer in 96 well round bottom tissue culture plates (Costar 3799, Corning inc., NY). GAS are incubated for 1 hour due to the poor ability of GAS to grow in such buffers. After incubation, the cells are diluted from 10× to 100,000×, and each of 20 ml of those solutions are plated in triplicate on tryptic soy broth (for S. aureus) and Todd Hewitt broth (for GAS and E. coli), and the mean number of colonies is determined. The number of cfu per ml is calculated, and the blocking activity of the examined test compounds to block the bactericidal activities of the AMP/AML will be calculated as follows: (cell survival after AMP/AML incubation)/(cell survival after incubation without AMP/AML)×100, which represents the percentage of cells that are alive, as compared to those which are not (cell survival after AMP/AML+test compound incubation)/(cell survival after incubation with test compound alone)×100.

All compounds identified will be screened for one or all of the following effects: their ability to inhibit the antimicrobial activity of the AMP to which they were raised against; their ability to affect the proliferation or differentiation or other cellular processes of cultured cells of the affected target tissue, originally isolated from normal or diseased individuals or models, for example HaCaT, primary human or murine keratinocytes or fibroblasts for screening for psoriasis; the effects of the inhibitors on activation of the immune system.

Identified compounds may be further screened for their effects on organotypic cocultures and animal models so as to identify inhibitors that will be able to effectively inhibit a desired biological effect or combination of biological effects. This may include, where suitable, identifying compounds that will inhibit the effects of AMPs/AMLs on proliferation/differentiation balance but which maintain their antibacterial/antimicrobial activity.

The test compound or regulator may be any of various type of molecule, such as a small synthetic/non-polypeptidic molecule.

The test compound or regulator may advantageously be a peptide, a protein or a glycosylated protein.

Test compounds and regulators of the present invention of any of various suitable types may be obtained from a commercial chemical library such as, for example, one held by a large chemical company such as Merck, Glaxo Welcome, Bristol Meyers Squib, Monsanto/Searle, Eli Lilly, Novartis, Pharmacia UpJohn, and the like. Test compounds and regulators of the present invention of any of various suitable types may also be ordered via the World Wide Web (Internet) via companies such as Chemcyclopedia (http://www.mediabrains.com/client/chemcyclop/BG1/search.asp). Alternatively, test compounds and regulators of the present invention of any of various suitable types may be synthesized de novo using standard chemical and/or biological synthesis techniques. Ample guidance for synthesis of molecules suitable for use as test compounds or regulators of the present invention of any of various suitable types is provided in the literature of the art. For biological synthesis of molecules, such as polypeptides and nucleic acids, refer, for example to: Sambrook et al., infra; and associated references in the Examples section which follows. For guidance regarding chemical synthesis of molecules, refer, for example to the extensive guidelines provided by The American Chemical Society (http://www.chemistry.org/portal/Chemistry). One of ordinary skill in the art, such as, for example, a chemist, will possess the required expertise for chemical synthesis of suitable test compounds.

In designing a small molecules capable of binding the AMP/AML, several features, such as structures of antibody, receptors, ligands, and relevant biochemical and biological data may be considered. Such features may include de novo folding design using energy minimization and molecular dynamics, and comparative modeling followed by energy minimization and molecular dynamics. These two approaches differ only in developing the trial or initial structures. The folding patterns are studied using energy minimization and molecular dynamics.

As used herein, the term “peptide” includes native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body or more capable of penetrating into target cells. Such modifications include, but are not limited to N terminus modification, C terminus modification, peptide bond modification, including, but not limited to, CH2-NH, CH2-S, CH2-S═O, O—C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992).

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 2 and 3 below list naturally occurring amino acids (Table 2) and non-conventional or modified amino acids (Table 3) which can be used with the present invention.

TABLE 2
Naturally occurring amino acids.

| Amino Acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| as above | | |

TABLE 3
Non-conventional or modified amino acids.

| Non-conventional amino acid | Code |
|---|---|
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-α-methylalnine | Dnmala |
| D-α-methylarginine | Dnmarg |
| D-α-methylasparagine | Dnmasn |
| D-α-methylasparatate | Dnmasp |
| D-α-methylcysteine | Dnmcys |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | mser |
| L-α-methylvaline | Mtrp |
| L-α-methylleucine | Mval Nnbhm |
| N-(N-(2,2-diphenylethyl) | Nnbhm |
| carbamylmethyl-glycine | |
| 1-carboxy-1-(2,2-diphenyl | Nmbc |
| ethylanaino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgin |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcyclopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cyclododeclglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylserine | Dnmser |
| D-N-methylthreonine | Dnmthr |
| N-(1-methylethyl)glycine | Nva |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| N-(3-guanidinopropyl)glycine | Narg |
| N-(1-hydroxyethyl)glycine | Nthr |
| N-(hydroxyethyl)glycine | Nser |
| N-(imidazolylethyl)glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl(1)glycine | |

The peptides of the present invention can be utilized in a linear or cyclic form.

A peptide can be either synthesized in a cyclic form, or configured so as to assume a cyclic structure under suitable conditions.

For example, a peptide according to the teachings of the present invention can include at least two cysteine residues flanking the core peptide sequence. In this case, cyclization can be generated via formation of S—S bonds between the two Cys residues. Side-chain to side chain cyclization can also be generated via formation of an interaction bond of the formula —(—CH2-)n-S—CH-2-C—, wherein n=1 or 2, which is possible, for example, through incorporation of Cys or homoCys and reaction of its free SH group with, e.g., bromoacetylated Lys, Orn, Dab or Dap. Furthermore, cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH2)n-COOH)—C(R)H—COOH or H—N((CH2)n-COOH)—C(R)H—NH2, wherein n=1-4, and further wherein R is any natural or non-natural side chain of an amino acid.

Depending on the application and purpose, any of various AMPs/AMLs may be employed and/or regulated so as to practice the various embodiments of the present invention. Numerous examples of AMPs/AMLs suitable for use in the present invention are listed on the Internet/World Wide Web at http://www.bbcm.units.it/˜tossi/pag1.htm, and are described hereinbelow.

Examples of AMPs/AMLs include defensins, cathelicidins, and thrombocidins (alternately termed “platelet microbicidal proteins [PMPs]”).

Examples of defensins include alpha-defensins, beta-defensins, and neutrophil defensins.

Examples of alpha-defensins include alpha-defensin-1 to -6 (Mol Immunol. 2003 November;40(7):463-7; J Clin Invest. 1985 October;76(4):1427-35).

Examples of beta-defensins include beta-defensin-1 (Genomics. 1997 Aug. 1;43(3):316-20; Biochem Biophys Res Commun. 2002 Feb. 15;291(1):17-22; FEBS Lett. 1995 Jul. 17;368(2):331-5; Paulsen F et al., J Pathol. 2002 November;198(3):369-77), beta-defensin-2 (Biochemistry. 2001 Apr. 3;40(13):3810-6; Gene. 1998 Nov. 19;222(2):237-44; Paulsen F et al., J Pathol. 2002 November;198(3):369-77), beta-defensin-3 (Cell Tissue Res. 2001 November;306(2):257-64; J Biol Chem. 2002 Mar. 8;277(10):8279-89. Epub 2001 Dec. 11; J Biol Chem. 2001 Feb. 23;276(8):5707-13; Paulsen F et al., J Pathol. 2002 November;198(3):369-77), beta-defensin-4 (J Immunol. 2002 Sep. 1;169(5):2516-23), beta-defensin-5 (Am J Pathol. 1998 May,152(5):1247-58; J Biol Chem. 1992 Nov. 15;267(32):23216-25), and beta-defensin-6 (FEBS Lett. 1993 Jan. 4;315(2):187-92; Crit Care Med. 2002 February;30(2):428-34).

Beta-defensins include those encoded by five conserved beta-defensin gene clusters identified using a computational search strategy (Schutte B C. et al., 2002. Proc Natl Acad Sci USA. February 19;99(4):2129-33).

Examples of neutrophil defensins include neutrophil alpha-defensins and neutrophil beta-defensins.

Examples of neutrophil alpha-defensins include neutrophil alpha-defensin-1//human neutrophil peptide (HNP)-1 (J Clin Invest. 1985 October;76(4):1436-9; Paulsen F et al., J Pathol. 2002 November;198(3):369-77), neutrophil alpha-defensin-2/HNP-2 (J Clin Invest. 1985 October;76(4):1436-9; Paulsen F et al., J Pathol. 2002 November;198(3):369-77), neutrophil alpha-defensin-3/HNP-3 (J Clin Invest. 1985 October;76(4):1436-9; Paulsen F et al., J Pathol. 2002 November;198(3):369-77), neutrophil alpha-defensin-4/HNP-4 (Mol Immunol. 2003 November;40(7):463-7), human defensin-5 (HD-5; D. E. Jones and C. L. Bevins, J. Biol. Chem. 267 (1992), pp. 23216-23225; J Biol Chem. 1992 Nov. 15;267(32):23216-25; Mol Immunol. 2003 November;40(7):469-75; Quayle A J et al., Am. J. Pathol. 1998, 152:1247-1258; FEBS Lett. 1993 Jan. 4;315(2):187-92; D. E. Jones and C. L. Bevins, FEBS Lett. 315 (1993); Paulsen F et al., J Pathol. 2002 November;198(3):369-77), and human defensin-6 (HD-6; Mol Immunol. 2003 November;40(7):463-7), human defensin-5 (HD-5; D. E. Jones and C. L. Bevins, J. Biol. Chem. 267 (1992), pp. 23216-23225; J Biol Chem. 1992 Nov. 15;267(32):23216-25; Mol Immunol. 2003 November;40(7):469-75; Quayle A J et al., Am. J. Pathol. 1998, 152:1247-1258; FEBS Lett. 1993 Jan. 4;315(2):187-92; D. E. Jones and C. L. Bevins, FEBS Lett. 315 (1993); Paulsen F et al., J Pathol. 2002 November;198(3):369-77).

Examples of cathelicidins include LL-37/hCAP18 (LL-37) in humans (Curr Drug Targets Inflamm Allergy. 2003 September;2(3):224-31; Eur J Biochem. 1996 Jun. 1;238(2):325-32; Paulsen F et al., J Pathol. 2002 November;198(3):369-77). LL-37 is a 37 amino acid residue peptide corresponding to amino acid residue coordinates 134-170 of its precursor hCAP18/human cathelicidin antimicrobial peptide protein (GenBank: ACCESSION NP_{—}004336; VERSION NP_{—}004336.2 GI:39753970; REFSEQ: accession NM_{—}004345.3). The proliferation and angiogenesis pathway of LL-37 can be inhibited using pertussis toxin, an inhibitor of G-protein coupled receptors (Koczulla, R. et al., 2003. J. Clin. Invest 111:1665-1672). Similar AMPs/AMLs are listed in the following patent applications: US 2003120037, US 200309626, US20020141620, US20020507, CA 2383172, US 20020072495 and are incorporated by reference herein. The human antibacterial cathelicidin precursor hCAP-18, is synthesized in myelocytes and metamyelocytes and localizes to specific granules in neutrophils (Blood. 1997 Oct. 1;90(7):2796-803).

Examples of AMP-like molecules include chemokines or fragments thereof.

Examples of such chemokines include CC chemokines and CXC chemokines. Considerable overlap of chemokine and AMP functions has been demonstrated (Cole et al., 2001. J. Immunol. 167:623), and certain chemokines and defensins have actually been shown to bind to the same chemokine receptor, CCR6. Defensins and certain chemokines strikingly share similar characteristics, including size, disulfide bonding, interferon (IFN) inducibility, cationic charge, and more. Relevant similarities between chemokines and AMPs are described in the literature (refer, for example, to Durr and Peschel, 2002. Infection and Immunity 70:6515). As such various chemokines and antibodies specific for such chemokines may be employed in various applications of the present invention.

Examples of such CC chemokines include CCL1, CCL5/RANTES (Infect Immun. 2002 December;70(12):6524-33; Eur J Biochem 1996 Apr. 1;237(1):86-92), CCL8, CCL11, CCL17, CCL18, CCL19, CCL20/activation-regulated chemokine (LARC)/macrophage inflammatory protein-3 alpha (MIP-3 alpha)/Exodus-1/Scya20 (Yang D et al., Journal of Leukocyte Biology Volume 74, September 2003;74(3):448-55), CCL21, CCL22, CCL25, CCL27/CTACK, and CCL28 (J Biol Chem. 2000 Jul. 21;275(29):22313-23; J Immunol. 2003 Feb. 1;170(3):1452-61). CCL chemokines are described in Yang D et al., Journal of Leukocyte Biology Volume 74, September 2003; 74(3):448-55.

Examples of such CXC chemokines include CXCL1, CXCL2, CXCL3, CXCL4 (PF-4), CXCL7/NAP-2, CXCL8/IL-8, CXCL9 (MIG; Yang D et al., Journal of Leukocyte Biology Volume 74, September 2003; 74(3):448-55), CXCL10/IP-10 (The Journal of Immunology, 2001, 167: 623-627), CXCL11/IP-9/1-TAC (The Journal of Immunology, 2001, 167: 623-627), CXCL12/SDF-1 (Yang D et al., Journal of Leukocyte Biology Volume 74, September 2003; 74(3):448-55), CXCL13, CXCL14, connective tissue activating peptide 3 (CTAP-3; Infect Immun. 2002 December;70(12):6524-33; Eur J Biochem 1996 Apr. 1;237(1):86-92), and CTAP-3 precursor platelet basic protein. CXC chemokines are described in Yang D et al., Journal of Leukocyte Biology Volume 74, September 2003; 74(3):448-55.

Examples of fibrinopeptides include fibrinopeptide-A (Infect Immun. 2002 December;70(12):6524-33; Eur J Biochem 1996 Apr. 1;237(1):86-92), fibrinopeptide-B (Infect Immun. 2002 December;70(12):6524-33; Eur J Biochem 1996 Apr. 1;237(1):86-92).

Examples of AMPs/AMLs further include XCL1 (Yang D et al., Journal of Leukocyte Biology Volume 74, September 2003; 74(3):448-55), MIP-1beta (Yang D et al., Journal of Leukocyte Biology Volume 74, September 2003; 74(3):448-55).

Further examples of AMPs/AMLs include adrenomedullin (Regul Pept. 2003 Apr. 15;112(1-3):147-52; J Biol Chem 1998 Jul. 3;273(27):16730-8), alpha-melanocyte stimulating hormone (Cutuli M et al., J Leukoc Biol. 2000 February;67(2):233-9; Neuroimmunomodulation-2002-2003; 10(4):208-16), an angiogenin (Nature Immunology, March 2003), angiogenin-4 (Nature Immunology, March 2003), antibacterial peptides B/enkelytin (Neuroimmunol 2000 Sep. 22;109(2):228-35), antileukoprotease (ALP; Biochem Biophys Res Commun. 1998 Jul. 30;248(3):904-9; Am J Respir Crit Care Med 1999 July;160(1):283-90), a lymphokine-activated killer cell-derived antimicrobial peptide, a platelet-derived antimicrobial peptide, antimicrobial peptide PR39, an apolipoprotein, an apolipoprotein-C, apolipoprotein-C2 (Hypertens Pregnancy 2002; 21(3):199-204; Peptides. 2000 March;21(3):327-30), apolipoprotein-C3 (Hypertens Pregnancy 2002; 21(3):199-204; Peptides. 2000 March;21(3):327-30), an apolipoprotein-E (Hypertens Pregnancy 2002; 21(3):199-204; Peptides. 2000 March;21(3):327-30), apolipoprotein-E2 (Brain Res 1997 Feb. 21;749(1):135-8; Biochemistry 2002 Oct. 1;41(39):11820-3; Eur J Clin Chem Clin Biochem 1997 August;35(8):581-9), a bactericidal/permeability-increasing protein (Paulsen F et al., J Pathol. 2002 November;198(3):369-77; Mol Microbiol 1995 Aug. 17:523-31; J Biol Chem 1987 Nov. 5;262(31):14891-4), a bone morphogenetic protein (BMP), BMP-2/4, BMP-5, buforin, calcitermin (FEBS Lett. 2001 Aug. 24;504(1-2):5-10), a cathepsin, cathepsin B, cathepsin G, cathepsin K, a lysosomal cathepsin, a chromogranin (Blood 2002 Jul. 15;100(2):553-9), chromogranin A (Blood 2002 Jul. 15;100(2):553-9), chromogranin B (Blood 2002 Jul. 15;100(2):553-9), chymase (Immunology 2002 April;105(4):375-90), connective tissue activating peptide-3, cystatin (APMIS. 2003 November;111(11):1004-1010; Biol Chem Hoppe Seyler 1988 May;369 Suppl:191-7), DCD-1 (J Immunol Methods. 2002 Dec. 1;270(1):53-62), dermicidin (Nat Immunol. 2001 December;2(12):1133-7), elastase-specific inhibitor/SKALP (skin-derived antileucoproteinase)/elafin (Biochem Soc Trans. 2002 April;30(2):111-5; J Invest Dermatol 2002 July;119(1):50-5), eNAP-1, eosinophil cationic protein (Peptides. 2003 April;24(4):523-30; J Immunol 2002 March 168:2356-64; Eur J Biochem 1996 Apr. 1;237(1):86-92; Peptides. 2003 April;24(4):523-30; J Exp Med 1989 Jul. 1;170(1):163-76), ESC42, ESkine, FALL-39 (Proc Natl Acad Sci USA. 1995 Jan. 3;92(1):195-9), Fas ligand (FasL; Berthou C et al., J Immunol. 1997 Dec. 1;159(11):5293-300), fractalkine, a glycosaminoglycan, granulysin (Reprod Biol Endocrinol. 2003 Nov. 28; J Immunol. 2003 Mar. 15;170(6):3154-61; Cancer Immunol Immunother. 2002 January;50(11):604-14. Epub 2001 November; Expert Opin Investig Drugs. 2001 February;10(2):321-9), granzyme B (Berthou C et al., J Immunol. 1997 Dec. 1;159(11):5293-300), HAX-1, heparin binding protein/CAP37 (Paulsen F et al., J Pathol. 2002 November;198(3):369-77; J Clin Invest 1990 May;85(5):1468-76), a hepcidin (J Biol Chem. 2001 Mar. 16;276(11):7806-10. Epub 2000 Dec. 11; Eur J Biochem 2002 April 269:2232-7), an HE2, HE2alpha (Biol Reprod. 2002 September;67(3):804-13), an HE2alpha C-terminal fragment (Biol Reprod. 2002 September;67(3):804-13), HE2beta1 (Biol Reprod. 2002 September;67(3):804-13), an HE2-gene derived transcript, histatin (Antimicrob Agents Chemother 2001 December 45:3437-44; Biochem Cell Biol. 1998; 76(2-3):247-56), a histone, histone H2A, histone H-2b (Peptides. 2003 April;24(4):523-30; J Immunol 2002 March 168:2356-64; Eur J Biochem 1996 Apr. 1;237(1):86-92), HMG-17, HtpG, an HtpG homolog, HS1 binding protein, interleukin-8, lactoferrin (Eur J Nucl Med. 2000 March;27(3):292-301; Paulsen F et al., J Pathol. 2002 November;198(3):369-77; J Mammary Gland Biol Neoplasia 1996 July;1(3):285-95), a lymphokine-activated killer (LAK) cell AMP (Hua Xi Yi Ke Da Xue Xue Bao 2002 January;33(1):87-90), lysozyme (Paulsen F et al., J Pathol. 2002 November;198(3):369-77; Anat Embryol (Berl) 2002 July;205(4):315-23), a macrophage inflammatory protein (MIP), MIP-1 alpha, MIP-1beta, MIP-3alpha, a mast cell granule serine proteinase (Immunology 2002 April;105(4):375-90), a matrix metalloproteinase (MMP), MMP-2, MMP-7 (Paulsen F et al., J Pathol. 2002 November;198(3):369-77), migration inhibitory factor (J Immunol. 1998 Sep. 1;161(5):2383-90; Scand J Infect Dis. 2003; 35(9):573-6), MMP-9, MRP8 (Behring Inst Mitt. 1992 April;(91):126-37), MRP14 (Behring Inst Mitt 1992 April;(91):126-37), neutrophil gelatinase-associated lipocalin (NGAL; Exp Dermatol. 2002 December;11(6):584-91; Mol Cell. 2002 November;10(5):103343), neutrophil lysozyme (Int J Antimicrob Agents. 1999 September;13(1):47-51), an opioid peptide, perforin (Berthou C et al., J Immunol. 1997 Dec. 1;159(11):5293-300), phospholipase A(2) (PLA(2); Peptides. 2003 April;24(4):523-30; J Exp Med 1989 Jul. 1;170(1):163-76), platelet basic protein (Infect Immun. 2002 December;70(12):6524-33; Eur J Biochem 1996 Apr. 1;237(1):86-92), platelet factor-4, psoriasin (J Histochem Cytochem. 2003 May;51(5):675-85; Gläser R et al., J Invest Dermatol 117: 768(abstr 015)), retrocyclin (Proc Natl Acad Sci USA 2002 Feb. 19;99(4):1813-8), secretory leukocyte proteinase inhibitor (SLPI; Shugars D C et al., Gerontology. 2001 September-October;47(5):246-53; Biochem Soc Trans. 2002 April;30(2):111-5; J Invest Dermatol 2002 July;119(1):50-5), secretory phospholipase A(2) (Peptides. 2003 April;24(4):523-30; J Immunol 2002 March 168:2356-64; Eur J Biochem 1996 Apr. 1;237(1):86-92; Paulsen F et al., J Pathol. 2002 November;198(3):369-77), substance P, an S100 calcium-binding protein, S100A7, S100A8, S100A9, a thymosin, thymosin beta-4 (Infect Immun. 2002 December;70(12):6524-33; Eur J Biochem 1996 Apr. 1;237(1):86-92; Infect Immun. 2002 December;70(12):6524-33; Eur J Biochem 1996 Apr. 1;237(1):86-92), thymus and activation-regulated chemokine (TARC), TL1A, tryptase (Immunology 2002 April;105(4):375-90), ubiquicidin (Eur J Nucl Med. 2000 March;27(3):292-301; Hiemstra P S, van den Barselaar M T et al., J Leukocyte Biol 1999; 66: 423-428; J Nucl Med 2001 May 42:788-94), and urokinase-type plasminogen activator.

The AMP/AML may any one of 28 potential candidates for defensin like peptides which were computationally discovered. (Am J Respir Cell Mol Biol. 2003 July;29(1):71-80).

As described hereinabove, the present invention can be used to treat any of various diseases which are associated with: (i) a tumor; (ii) inflammation; (iii) an epithelial wound; (iv) dysregulation of growth/differentiation of a cell/tissue; (v) dysregulation of growth/differentiation balance of a cell/tissue; and/or (vi) angiogenesis.

Examples of diseases which can be treated according to the present invention are listed in U.S. patent application Ser. No. ______.

Examples of diseases which can be treated according to the present invention are also as follows.

Examples of tumors include a skin tumor, a keratinocytic tumor, a gastrointestinal tumor, a carcinoma, a melanoma, a squamous cell tumor, oral squamous cell carcinoma, lymphoma, a malignant tumor, a benign tumor, a solid tumor, a metastatic tumor and a non-solid tumor.

The concentration of human beta-defensin-2 in oral squamous cell carcinoma is much higher than in normal oral epithelium (Sawaki, K. et al., 2002. Anticancer Res. 22:2103-2107). There is a genetic link between proliferation of cells and cancer. Impairment of regulation of proliferation and differentiation lead to cancer development. A developing tumor needs help from neighboring cells in order to become cancerous. Overexpression or overactivity of cytokines is involved in orchestrating these processes. Continuous assault by chronic inflammation contributes to the transformation of cells as well. Angiogenesis is an important process for cancer development. AMPs are inductors of angiogenesis (Koczulla, R. et al., 2003. J. Clin. Invest 111:1665-1672). Therefore inhibiting differentiation and proliferation as well as angiogenesis by antagonists to AMPs and cytokines can be used to treat cancer. Urokinase-type plasminogen activator (uPA), has antimicrobial properties (Gyetko, M R. et al., 2002. J. Immunol. 168:801-809) and is involved in metastatic spreading of malignant cells. The in vitro and in vivo findings suggest that alpha-defensins are frequent peptide constituents of malignant epithelial cells in renal cell carcinoma with a possible direct influence on tumor proliferation (Muller, C A. et al., 2002. Am. J. Pathol. 160:1311-1324). Certain anti-angiogenic compounds were found to have potent anticancer property in vivo experimental studies. Therefore inhibition of angiogenic AMPs such as LL-37 is one form of treatment for cancer. Matrix metalloproteinases (MMPs) are known to play an important role in extracellular matrix remodeling during the process of tumor invasion and metastasis. Overexpression of MMP-2 and MMP-9 proteins was observed in a large percentage of ESCC tumors, respectively localized in tumor cell cytoplasm and stromal elements (J Cancer Res Clin Oncol. 2003 Oct. 16).

BMP-2/4 and BMP-5 but not BMPR-IA might be involved in the metastasis of oral carcinoma cells (Overexpression of BMP-2/4, -5 and BMPR-IA associated with malignancy of oral epithelium Oral Oncol. 2001, 37:225-33.)

Examples of diseases associated with an epithelial wound include a healing deficiency, an ulcer, a skin ulcer, a bed sore, a gastric ulcer, a peptic ulcer, a buccal ulcer, a nasopharyngeal ulcer, an esophageal ulcer, a duodenal ulcer, and a diabetes related healing deficiency.

Examples of diseases include an idiopathic/inflammatory disease, a chronic/inflammatory disease, an acute/inflammatory disease, an inflammatory/cutaneous disease, an inflammatory/gastrointestinal disease, a tumor associated with inflammation, an allergic disease, an autoimmune disease, an infectious disease, a malignant disease, a transplantation related disease, an inflammatory/degenerative disease, an injury associated with inflammation, a disease associated with a hypersensitivity, an inflammatory/cardiovascular disease, an inflammatory/glandular disease, an inflammatory/hepatic disease, an inflammatory/neurological disease, an inflammatory/musculo-skeletal disease, an inflammatory/renal disease, an inflammatory/reproductive disease, an inflammatory/systemic disease, an inflammatory/connective tissue disease, an inflammatory/neurodegenerative disease, necrosis, an inflammatory disease associated with an implant, an inflammatory/hematological disease, an inflammatory/eye disease, an inflammatory/respiratory disease.

Examples of cutaneous/inflammatory diseases include psoriasis, dandruff, pemphigus vulgaris, lichen planus, atopic dermatitis, scleroderma, dermatomyositis, alopecia, blepharitis, skin carcinoma, melanoma, squamous cell carcinoma, acne vulgaris, erythema toxicum neonatorum, folliculitis, skin wrinkles, autoimmune bullous skin disease, bullous pemphigoid, pemphigus foliaceus, dermatitis, and drug eruption.

Dandruff can be classed as an inflammatory, abnormal proliferative or abnormal differentiation disease whereby flaky skin on the scalp protrudes, as with psoriasis, due to abnormalities in proliferation/differentiation balance caused by over reactivity of AMPs such as LL-37 and the defensins. Statistical surveys in Swedish population showed a correlation between dandruff and psoriasis. People with a genetic (in-the-family) risk of developing psoriasis have a significantly higher proportional rate of dandruff sufferers. Therefore the psoriasis treatment method described by the present invention is applicable to treatment of psoriasis.

Examples of gastrointestinal/inflammatory diseases include Crohn's disease, chronic autoimmune gastritis, autoimmune atrophic gastritis, primary sclerosing cholangitis, autoimmune achlorhydra, colitis, ileitis, chronic inflammatory intestinal disease, inflammatory bowel syndrome, chronic inflammatory bowel disease, celiac disease, an eating disorder, gallstones and a gastrointestinal ulcer.

Crohn's disease is an inflammatory bowel disease. Since the bowel is exposed to the outer environment, the importance of AMPs as part of its defense and normal cellular regulation is important, as in skin, and the activity of the AMPs plays an important role in the normal physiology as well as pathological conditions in these tissues. Abnormalities in the expression and/or activity of the AMPs will contribute to pathologies in these tissues. Paneth cells (a specific type of cell in the intestine) are required to help promote normal vessel formation in cooperation with bacteria—mice absent Paneth cells were incapable of appropriate blood vessel formation. Of note, colonization by one particular type of bacteria commonly found in normal mouse and human intestine, called Bacteroides thetaiotaomicron, or B. thetaiotaomicron, stimulated blood vessel development as efficiently as implantation of a whole microbial society. The conclusion, B. thetaiotaomicron and Paneth cells work together to stimulate postnatal blood vessel formation. The ability of AMPs to act as chemoattractants for cells of the innate- and adaptive-immune system plays an important role in perpetuating chronic inflammation in the gastrointestinal tract (Cunliffe, R N, Mahida, Y R., 2003. J Leukoc Biol. October 2 [Epub ahead of print]). The AMP LL-37, beta-defensins, human alpha-defensins, beta-defensins (including HD5), HN-6, lysozyme and secretory PLA2, TL1A, are expressed in Paneth cells and intestine, secretory epithelial cells in the small intestine (Ghosh, D. et al., 2002. Nat. Immunol. 3:583-590; Fellermann, K. et al., 2003. Eur. J. Gastroenterol. Hepatol. 15:627-634). Where alpha-defensins are overexpressed, they are chemoattract naive T and immature dendritic cells and dendritic cells and monocytes (Yang, D. et al., 2000. J. Leukoc. Biol. 68:9-14; Risso, A., 2000. J. Leukoc. Biol. 68:785-792; Territo, M C. et al., 1989. J. Clin. Invest 84:2017-2020). Human alpha-defensins as well as other AMPs contribute to local intestinal host defense as part of innate immunity and may be of major relevance in microbial infection and chronic inflammatory bowel disease (Wehkamp, J. et al, 2002. Dig. Dis. Sci. 47:1349-1355). The alpha-defensins convert an acute inflammation to a chronic inflammation by downregulating human polymorphonuclear leukocyte chemotaxis, for example, alpha-defensin-1/human neutrophil protein-1, acts as an antichemotactic agent for human polymorphonuclear leukocytes). It is known that chronic inflammation is commonly characterized by the presence of increased cell proliferation and connective tissue than exudate with the presence of lymphocytes and plasma cells rather than polymorphonuclear leukocytes. Thus, suitable regulation of such AMPs/AMLs can be used to treat diseases such as inflammatory bowel disease, Crohn's disease and ulcerative colitis.

Gastritis is an inflammatory condition of the stomach. There are two main forms of gastritis, A and B. Gastritis type A is considered to develop in an autoimmune process. In both types there is a role for infectious agents such as Helicobacter pylori. AMPs are involved in both processes. Defensins are involved in pathogenesis of gastritis (Bajaj-Elliott, M. et al., 2002. Gut 51:356-361). Thus, suitable regulation of such AMPs/AMLs can be used to treat diseases such as gastritis.

Examples of allergic/inflammatory diseases include asthma, hives, urticaria, a pollen allergy, a dust mite allergy, a venom allergy, a cosmetics allergy, a latex allergy, a chemical allergy, a drug allergy, an insect bite allergy, an animal dander allergy, a stinging plant allergy, a poison ivy allergy, anaphylactic shock, anaphylaxis, atopic allergy and a food allergy.

Examples of hypersensitivity include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

Examples of cardiovascular/inflammatory and/or inflammatory/hematological diseases include atherosclerosis, Takayasu's arteritis, polyarteritis nodosa, Raynaud's phenomenon, temporal arteritis, inflammatory anemia, inflammatory lymphopenia, pernicious anemia, occlusive disease, myocardial infarction, thrombosis, Wegener's granulomatosis, lymphoma, leukemia, Kawasaki syndrome, anti-factor VIII autoimmune disease, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease, iron-deficiency anemia, and anti-helper T lymphocyte autoimmunity.

Inflammation is part of the pathological process leading to the development of atherosclerosis. Chlamydia pneumonia as well as other various microorganisms serve as potential etiological factors, linking inflammation and atherosclerosis. Inflammation is a predisposing factor as well as a consequence of several CNS pathologies. Inflammation is part of the pathophysiologic processes occurring after the onset of cerebral ischemia in ischemic stroke, as well as other CNS pathologies such as head injury and subarachnoid hemorrhage. In addition, inflammation in the CNS or in the periphery by itself is considered as a risk factor for the triggering the development of cerebral ischemia Endothelial cells express and secrete AMPs. Cationic antimicrobial protein of 37 kDa (CAP37) also termed heparin binding protein, originally isolated from human neutrophils, is an important multifunctional inflammatory mediator is expressed within the vascular endothelium associated with atherosclerotic plaques (Lee, T D. et al., 2002. Am. J. Pathol. 160:841-848). Human beta-defensin-2 is expressed by astrocytes and its expression is increased in response to cytokines and LPS (Hao, H N. et al., 2001. J. Neurochem. 77:1027-1035). Therefore, AMP inhibition can be used for treatment or prevention of these conditions.

Anemia associated with inflammatory/chronic diseases is one of the body's methods of fighting pathogens by reducing available inter cellular iron uptake of pathogens. Iron is absorbed by neutrophils. Sometimes chronic inflammation can occur without the presence of pathogens. Under chronic inflammatory conditions, cytokines induce a diversion of iron traffic leading to hypoferremia. Such as in chronic bacterial endocarditis, osteomyelitis, juvenile rheumatoid arthritis, rheumatic fever, Crohn's disease, and ulcerative colitis and Chronic renal failure. Transferrin bound iron transports to monocytes causing anemia This “transport” is thought to be related to AMP activity. Cytokines IL-1, IL-6 and TNF-beta initiate defensin production and defensin initiate the cytokine production, the result being iron over absorption by monocytes. The regulation of iron transport by cytokines is a key mechanism in the pathogenesis of anemia of chronic disease (Ludwiczek, S. et al., 2003. Blood 101:4148-4154). Therefore, regulation of AMPs can be used to regulate iron level homeostasis. Hepcidin AMP is known to regulate iron uptake, therefore inhibiting hepcidin can be used to increase iron absorption (Nicolas, G. et al., 2002. Blood Cells Mol. Dis. 29:327-335). However, there are other AMPs indirectly involved in iron regulation such as defensin and LL-37. Since HNP-1 is a non-specific defensive peptide present in neutrophils, it plays an important role in the protection against diseases such as oral lichen planus, leukoplakia, and glossitis associated with iron deficiency (Mizukawa, N. et al., 1999. Oral Dis. 5:139-142). Likewise all cationic neutrophil derived AMPs would induce iron hypoferremia when over expressed. Therefore inhibition of these AMPs can be used to treat such diseases.

Leukocyte SLPI (secretory leukocyte proteinase inhibitor (SLPI)) expression seems to be up-regulated in active Wegner's granulomatosis, therefore inhibiting its activity can be used to treat diseases such as Wegener's granulomatosis and other types of vasculitis

Examples of glandular/inflammatory diseases include type I diabetes, type II diabetes, type B insulin resistance, Schmidt's syndrome, Cushing's syndrome, thyrotoxicosis, benign prostatic hyperplasia, pancreatic disease, Hashimoto's thyroiditis, idiopathic adrenal atrophy, Graves' disease, androgenic alopecia, thyroid disease, thyroiditis, spontaneous autoimmune thyroiditis, idiopathic myxedema, ovarian autoimmunity, autoimmune anti-sperm infertility, autoimmune prostatitis, Addison's disease, and Type I autoimmune polyglandular syndrome.

Diabetes mellitus is a systemic disease with several major complications affecting both the quality and length of life. One of these complications is periodontal disease (periodontitis). Periodontitis is much more than a localized oral infection. (Iacopino, A M., 2001. Ann. Periodontol. 6:125-137). When diabetes mellitus is under therapeutic control, periapical and other lesions heal as readily as in nondiabetics (Bender, I B, Bender, A B. et al., 2003. J. Endod. 29:383-389). Recent studies on diseases which involve insulin insensitivity (e.g. obesity, type 2 diabetes and atherosclerosis) also show increased cytokine production and markers of inflammation. Evidence at present favors chronic inflammation as a trigger for chronic insulin insensitivity, rather than the reverse situation. (Grimble, R F., 2002. Curr. Opin. Clin. Nutr. Metab Care 5:551-559). Recent human studies have established a relationship between high serum lipid levels and periodontitis. Possible causes are a high glucose levels (such as hyperglycemia of diabetics) with added LDL levels such as in high diabetic patients are prone to elevated low density lipoprotein cholesterol and triglycerides (LDL/TRG) even when blood glucose levels are well controlled, lead to LPS-like bondings that induce AMP overexpression. Thus, the present invention can be used to treat diabetes and diabetes related diseases such as periodontitis and diabetes associated healing deficiencies.

Proliferative retinopathy is one of the chronic complications of diabetes. The process includes the development of abnormal blood vessels that might lead to retinal detachment and blindness. LL37 and other AMPs are involved in angiogenesis (Koczulla, R. et al., 2003. J. Clin. Invest 111:1665-1672), therefore antibodies and antagonists to LL-37 can be used to prevent the development of newly formed blood vessels and therefore for preventing diabetes related eye diseases.

Examples of hepatic/inflammatory diseases include primary biliary cirrhosis, active chronic hepatitis, lupoid hepatitis, autoimmune hepatitis, and hepatic cirrhosis.

Examples of neurological/inflammatory diseases include neurodegenerative disease, multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis, spongiform encephalopathy, migraine, headache, cluster headache, and stiff-man syndrome.

With respect to multiple sclerosis (MS), defensins and lactoferrins exist in cerebrospinal fluid (CSF). These peptides have antimicrobial expression in some diseases like pneumonia and meningitis, which may trigger a pathway. It seems that pathways to MS are similar to rheumatoid arthritis where AMPs reside in the synovial fluid surrounding the joint. Peptides involved are amongst others: IP-10, defensins and lactoferrins, CAP37.

There is a relationship between polymorphism at the apolipoprotein E2 Apo(a) locus relation to Alzheimer's disease (AD; Barbier, A. et al., 1997. Eur. J. Clin. Chem. Clin. Biochem. 35:581-589; Compton, D. et al., 2002. Neurosci. Lett. 331:60-62). ApoE has antimicrobial properties and therefore regulating this molecule can be use to treat Alzheimer's disease. Essentially, all polymorphisms of this peptide are somehow involved in the pathogenesis of the disease however the e4 isoform is more active. People with the e4/e4 genotype have the highest risk, but people with the e2/e4 or e3/e4 genotypes are also likely to develop the disease. While the APOE e4 allele defines a greater risk, the presence of e4 cannot alone predict the disorder prior to the onset of symptoms—only 40 percent of all Alzheimer's patients have the e4 allele. The e4 allele is also associated with higher cholesterol absorption which leads to higher cholesterol levels in the blood. The e4/e4 genotype is found in only 1-3 percent of the Westernized population. However, the probability that a Westernized individual with the e4/e4 genotype will develop Alzheimer's disease is 60 percent, with women at greater risk than men. For individuals who consume high-cholesterol diets, having the e4 allele may also increase the risk of coronary artery disease. Complex formation with ApoE enhances internalization of soluble Abeta uptake into terminals. LPS-induced astrogliosis in ApoE transgenic mice is regulated isoform-specifically by ApoE3 and not by ApoE4 and suggest that similar mechanisms may mediate the phenotypic expression of the ApoE4 genotype in AD and in other neurodegenerative diseases. Therefore inhibitors of the present invention specific for this protein polymorphism can prevent or delay the onset of Alzheimer disease (Rebeck, G W. et al., 2002. J. Alzheimers. Dis. 4:145-154). The beneficial non-rejected ApoE2 and E3 is introduced as a replacement (via injection or otherwise) in conjunction with the monoclonal antibody/antagonist to the “bad” isomer/isozyme/polymorphic protein at its specific site (the analogue) responsible for the onset of Alzheimer's disease (Baum, L. et al., 2000. Microsc. Res. Tech. 50:278-281). In AD but not in controls, the cerebral microcirculation expresses the inflammatory mediator AMP CAP37, the heparin binding protein (Grammas, P., 2000. Neurobiol. Aging 21:199-205). Antibody and antagonists to CAP37 can therefore also be used for treating Alzheimer's disease (Pereira, H A. et al., 1996. Neurobiol. Aging 17:753-759; Neurobiol Aging, 2002, 23:531-6). FPRL1, an LL-37 receptor therefore constitutes a molecular target for the development of therapeutic agents for Alzheimer Disease (Cui, Y. et al., 2002. J. Leukoc. Biol. 72:628-635). LL-37 acts in parallel with A(beta) peptides in activating the same G-protein-coupled chemoattractant receptor, FPR-Like-1 (Le, Y. et al., 2001. J. Neurosci. 21:RC123).

Examples of connective tissue/inflammatory diseases include arthritis, rheumatoid arthritis, pyogenic arthritis, mixed connective tissue disease, cholesteatoma, relapsing polychondritis, autoimmune myositis, primary Sjogren's syndrome, smooth muscle autoimmune disease, myositis, tendinitis, a ligament inflammation, chondritis, a joint inflammation, a synovial inflammation, carpal tunnel syndrome, osteoarthritis, ankylosing spondylitis, a skeletal inflammation, an autoimmune ear disease, osteoporosis, fibromyalgia, periodontitis, and an autoimmune disease of the inner ear.

With respect to diseases such as arthritis, AMPs are expressed and produced in healthy and inflamed human synovial membranes. Deposition of the AMPs lysozyme, lactoferrin, secretory phospholipase A(2) (sPA(2)), matrilysin (MMP7), human neutrophil alpha-defensin-1, -2, and -3, human beta-defensi-1, and human beta-defensin-2 was determined by immunohistochemistry. Expression of mRNA for the AMPs bactericidal permeability-increasing protein (BPI), heparin binding protein, LL37, human alpha-defensin-5, human alpha-defensin-6, and human beta-defensin-1, -2, and -3 was analyzed by reverse transcription-polymerase chain reaction (RT-PCR). RT-PCR revealed CAP37 and human beta-defensin-1 mRNA in samples of healthy synovial membrane. Additionally, human beta-defensin-3 and/or LL37 mRNA was detected in synovial membrane samples from patients with pyogenic arthritis (PA), osteoarthritis (OA) or rheumatoid arthritis (RA). Immunohistochemistry has identified lysozyme, lactoferrin, sPA(2), and MMP7 in type A synoviocytes of all samples. Human beta-defensin-1 was only present in type B synoviocytes of some of the samples. Immunoreactive human beta-defensin-2 peptide was only visible in some inflamed samples. HNP1-3 was detected in both healthy and inflamed synovial membranes. The data suggest that human synovial membranes produce a broad spectrum of AMPs. Under inflammatory conditions, the expression pattern changes, with induction of human beta-defensin-3 in PA (LL37 in RA; human beta-defensin-3 and LL37 in OA) as well as down-regulation of human beta-defensin-1 (Paulsen, F. et al., 2002. J. Pathol. 198:369-377; Cunliffe, R N, Mahida, Y R., 2003. J Leukoc Biol. October 2 [Epub ahead of print]). Thus blocking one or more of these proteins or their activity will inhibit the pathological process in a disease such as arthritis.

Microbial mixed keratin-biofilms in cholesteatomas are caused by AMPs which are overexpressed (Jung, H H. et al., 2003. Laryngoscope 113:432-435; Chole, R A, Faddis, B T., 2002 Arch. Otolaryngol. Head Neck Surg. 128:1129-1133), AMPs such as LL-37 or other defensins or other AMPs are involved. Therefore, suitable regulation of such AMPs can be used for treating diseases such as cholesteatomas.

Examples of inflammatory/renal diseases include diabetic nephropathy.

High glucose levels (such as hyperglycemia of diabetics) with added LDL levels such as in high diabetic patients are prone to elevated low density lipoprotein cholesterol and triglycerides (LDL/TRG) even when blood glucose levels are well controlled, and lead to LPS-like bondings that induce AMP overexpression. Overexpression of beta-defensin-1 mRNA plays a role in diabetic nephropathy (Page, R A, Malik, A N. et al., 2003. Biochem. Biophys. Res. Commun. 310:513-521). The cytotoxic activity of defensins can be correlated to the location of the inflammation in the kidney where defensins play a role in the pathogenesis of chronic glomerulonephritis and pyelonephritis (Rebenok, A Z. et al., 1999. Ter. Arkh. 71:62-67). Therefore inhibiting such AMPs can be used to treat diabetic nephropathy.

Examples of inflammatory/reproductive diseases include repeated fetal loss, ovarian cyst, or a menstruation associated disease.

Examples of inflammatory/systemic diseases include systemic lupus erythematosus, systemic sclerosis, septic shock, toxic shock syndrome, Reiter's syndrome, and cachexia.

Examples of inflammatory/infectious diseases include candidiasis, a fungal infection, mycosis fungoides, a chronic infectious disease, a subacute infectious disease, an acute infectious disease, a viral disease, a bacterial disease, a protozoan disease, a parasitic disease, a mycoplasma disease, gangrene, sepsis, a prion disease, influenza, tuberculosis, bacterial pneumonia, malaria, acquired immunodeficiency syndrome, chronic fatigue syndrome, and severe acute respiratory syndrome.

LL-37 binds to surface proteins of fungi and bacterial LPS. LL-37 is not hostile to fungi in saline solution (Turner, J. et al., 1998. Antimicrob. Agents Chemother. 42:2206-2214). Likewise, not all AMPs are hostile to Candida. Therefore inhibiting LL-37 and to other AMPs that are not hostile to fungi can be used to prevent the adhesion of fungi to cells, and hence to treat fungal diseases.

Examples of transplantation related/inflammatory diseases include graft rejection, chronic graft rejection, subacute graft rejection, acute graft rejection hyperacute graft rejection, rejection of an implant and graft versus host disease.

Examples of implants include a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

Examples of injury/inflammation include a skin wound, an abrasion, a bruise, a cut, a puncture wound, a laceration, an impact wound, a concussion, a contusion, a thermal burn, frostbite, a chemical burn, a sunburn, a desiccation, a radiation burn, a radioactivity burn, a smoke inhalation, a torn muscle, a pulled muscle, a torn tendon, a pulled tendon, a pulled ligament, a torn ligament, a hyperextension, a torn cartilage, a bone fracture, a pinched nerve and a gunshot wound.

Examples of inflammatory/respiratory diseases include asthma, allergic asthma, diffuse panbronchiolitis, emphysema, idiopathic pulmonary fibrosis, cystic fibrosis, influenza, sinusitis, sinusitis and chronic obstructive pulmonary disease.

The literature provides evidence that the present invention can be used to treat diseases such as asthma, chronic obstructive pulmonary disease, cystic fibrosis, and sinusitis. Inflammation is stimulated by AMPs (respiratory epithelial, endothelial, bronchus, larynx, kidney, fibroblast, and other endothelial cells). Furthermore, adherence of Haemophilus influenzae to bronchial epithelial cells is enhanced by neutrophil defensins, which are released from activated neutrophils during inflammation (Gorter, A D. et al., 1998. J. Infect. Dis. 178:1067-1074). Adherence of H. influenzae to various epithelial, fibroblast-like and endothelial cell types was significantly enhanced by defensins. Defensins stimulated also the adherence of Moraxella catarrhalis, Neisseria meningitidis and nonencapsulated Streptococcus pneumoniae (Gorter, A D. et al., 2000. FEMS Immunol. Med. Microbiol. 28:105-111), H. influenzae, M. catarrhalis, N. meningitidis and nonencapsulated, S. pneumoniae. The chronic inflammation of cystic fibrosis (CF) is associated with increased levels of AMPs in respiratory tract secretions. However, the CF airway surface fluid is diminished in its ability to kill bacteria This defect is reflected in chronic, high-level bacterial colonization and recurrent pneumonia with organisms such as P. aeruginosa. The bacteria-killing ability of CF airway fluid is restored when its salt concentration is lowered to normal levels, suggesting that the abnormally high salt concentrations produced by the defective CF transmembrane conductance regulator might be responsible. The bacterial killing ability of epithelial-derived AMPs such as the human beta-defensins and cathelicidin are inactivated by high salt concentrations, suggesting a defect in this component of innate immune defense might be responsible for the chronic pulmonary infections seen in CF patients. As with Candidiasis, AMPs in high saline solutions encourage pathogenic activity by enabling pathogens to cling on to cell surface membranes. High concentrations of defensins have been found in purulent airway secretions from patients with chronic obstructive pulmonary disease, cystic fibrosis, diffuse panbronchiolitis, increasing infection and disease progression. Antibodies to defensins 1-6 can therefore reduce infection and inflammation. M. pneumoniae infection contributes to the pathogenesis of chronic asthma at different levels of the airways by inducing the chemokine RANTES in small airways. Inhibition of RANTES is necessary. Thus, blocking the expression of these and other AMPs will be advantageous to halting the progression of the disease and to treatment. Intratracheal instillation of defensins causes acute lung inflammation and dysfunction, suggesting that high concentrations of defensins in the airways may play an important role in the pathogenesis of inflammatory lung diseases (Zhang, H. et al., 2001. Am. J. Physiol Lung Cell Mol. Physiol 280:L947-L954). They are overexpressed in cystic fibrosis, diffuse panbroncheolitis, idiopathic pulmonary fibrosis and acute respiratory distress syndrome, and in infectious diseases (Aarbiou, J. et al., 2002. Ann. Med. 34:96-101). In addition to their antimicrobial role, human neutrophil defensins also contribute to adaptive immunity by mobilizing T cells and dendritic cells (Yang, D. et al., 2000. J. Leukoc. Biol. 68:9-14).

Human beta-defensin-2 is expressed in nasal mucosa and is upregulated in a condition of chronic inflammation of the sinus (Chen, P H, Fang, S Y., 2003. Eur. Arch. Otorhinolaryngol. September 18′ [Epub ahead of print]). Therefore downregulation of human beta-defensin-2 can be used to treat diseases such as sinusitis.

Examples of inflammatory/eye diseases include dry-eye disease, phacogenic uveitis, blepharitis and sympathetic ophthalmia.

Dry eye disease is a chronic inflammatory eye disease. Is particularly an issue for post-menopausal women, the elderly, and patients with systemic diseases such as Sjogren's syndrome, rheumatoid arthritis, lupus and diabetes (37% of people with diabetes suffer from the disease and 28% of adults having the disease). Defensins act as chemokines to T-cells (Stern, M E, et al., 2002. Invest Ophthalmol. Vis. Sci. 43:2609-2614). Upregulation of AMP including human beta-defensin-2 is a feature of dry eye disease. Human beta-defensin-2 was expressed in conjunctival epithelium of patients with moderate dry eye (Narayanan, S. et al., 2003. Invest Ophthalmol. Vis. Sci. 44:3795-3801). Therefore inhibiting AMP/AML (especially human betadefensin-2) production and activity can be used to treat diseases such as dry-eye disease.

Erythema toxicum neonatorun is a common, inflammatory skin reaction in healthy newborn infants characterized by an accumulation of activated immune cells in the lesions. Its etiology and physiologic significance are still unclear. Recently strong staining for psoriasin was seen in the entire epidermal layer (Marchini, G. et al., 2003. Pediatric Dermatology 20:377-384). Thus, blocking this protein may be beneficial for treating the condition.

The majority of acne biopsies display a marked upregulation of defensin-2 immunoreactivity in the lesional and perilesional epithelium—in particular in pustules—and a less marked upregulation of defensin-1 immunoreactivity (Chronnell, C M T. et al., 2001. Journal of Investigative Dermatology 117:1120-1125). Folliculitis is a common skin disease with inflammation of the hair follicle, clinically manifested as papules and pustules. It was recently shown by immunohistochemistry that human neutrophil peptide (HNPs) and human beta-defensin-2 are abundantly present in the lesions of superficial folliculitis. Immunoreactivity for HNPs was observed in infiltrating PMN leukocytes and pustules in the interfollicular spaces. In contrast, immunoreactivity for human beta-defensin-2 was observed in the perilesional and lesional epidermis of the affected hair follicle. The distribution pattern of human beta-defensin-2 was similar to that in acne vulgaris lesions (Oono, T. et al., 2003. British Journal of Dermatology 148:188-191)

Increased levels of AMP expression appear also appear to be correlated with pathogenesis of diseases such as lichen planus which is associated with elevated levels of beta-defensin, and sarcoidosis which is associated with elevated levels of LL-37. Cathelicidin levels were found to be increased in inflammatory skin lesions of erythema toxicum neonatorum, and to apparently correlate with inflammatory/activated neutrophils, eosinophils, and dendritic cells. High levels of LL-37 have also been demonstrated in epidermis during pathogenesis of verruca vulgaris or condyloma accuminata.

Examples of the disease include aging and aging-related diseases. In the Examples section, below, it is shown that the keratinocyte proliferation/differentiation balance is affected by the concentration of antimicrobial peptides. Over expressing human beta-defensin-2 on 3-D organotypic skin co-culture models caused the unorganized proliferation of keratinocytes and fibroblasts. Aging of skin and other body tissue has many causes. One major contributor to wrinkles and aging in skin is the buildup of discrepancies and disorganization in collagen proteins. Fibroblasts are cells that produces the collagen matrix surrounding cells therefore increasing the density of fibroblasts implies increasing collagen output thereby rejuvenating skin. As described in the Examples section below, antibody to human beta-defensin-2 on 3-D organotypic skin co-culture models showed increased differentiation on account of proliferation of keratinocytes as well as increased proliferation of fibroblasts. This led to more dense fibroblast regions and a better organized skin than the untreated control (normal skin). Both keratinocytes and fibroblasts were organized better than the control untreated skin and there appeared a significant enlargement of normalized epidermal thickness. The result data below shows that AMPs regulate the differentiation/proliferation balance and in so doing their level of concentration in skin determines how well the cells are organized. This demonstrated the importance of AMPs and inhibitors to AMPs for fibroblasts, and therefore collagen as well, as for keratinocyte organization and for anti aging and anti wrinkle therapies for skin. This anti aging therapy should holds true for many body tissues where AMPs can be found.

Complexation or cross linking of fibrils and protofibrils with amps is involved in pathogenesis of age related diseases. As in other chronic inflammatory diseases AMP inhibitors can prevent inflammation and tissue morphological changes leading to diabetes, Alzheimer's disease, Parkinson's disease, and spongiform encephalopathies. The first stages of diabetes commence with high insulin levels leading to overexpression of AMPs which in turn results in morphological changes in pancreatic tissue. This results in systemic underexpression of insulin. In addition, protofibril complexation with AMPs is implicated in diseases such as age-related diseases like type II diabetes, Alzheimer's disease, Parkinson's disease, spongiform encephalopathies and other prion diseases, and type II diabetes. Clumps of misfolded proteins known as amyloid fibrils are involved in killing cells in such diseases. Smaller structures (protofibrils) formed prior to the mature fibrils, are more likely to get through the cell membrane, and may therefore be the more toxic than fibrils. Therapeutic efforts have focused on breaking up these deposits. In Alzheimer's disease they are called amyloid plaques; in Parkinson's disease they are called Lewy bodies; in type II diabetes they are called islet amyloid deposits and occur in the “islets of Langerhans,” the area of the pancreas where insulin is produced and regulated. Type II diabetes is one of the most common amyloid-related diseases. Inhibiting the earlier stage of protofibril formation is essential to preventing age related diseases. It has been shown that chronic inflammatory diseases, like Alzheimer's disease for example, involve colocalization of AMPs with amyloid plaques (Pereira, H A. et al., 1996. Neurobiol. Aging 17:753-759). It is postulated that AMPs form complexes with the protofibrils. Complex formation is controlled by a finely balanced interplay of hydrophobic and electrostatic interactions with none of these two interactions alone being strong enough to ensure complexation under these polar conditions. Evidence for this has been demonstrated where the highly cationic small protein defensin was isolated along with the amyloid A protein from the fibrils (Liepnieks, J J. et al., 1995. Biochim. Biophys. Acta 1270:81-86). Likewise the antimicrobial protein ApoE4 isoform has an associated higher risk for Alzheimer and is also the most cationic of all the differing isoforms of ApoE differing from ApoE3 by one charge unit and from ApoE2 by two (Mahley, R W, Rall, S C, Jr., 2000. Annu. Rev. Genomics Hum. Genet. 1:507-537; Castano, E M. et al., 1995. J. Biol. Chem. 270:17610-17615). The present inventors hypothesize that AMPs (being amongst other things highly cationic and small peptides) act as catalysts or as cross linking initiators with amyloid protein, in light of the close ultrastructural relationship between sulfated proteoglycans and AA amyloid fibrils (Snow A D. et al., 1987. Lab Invest. 57:687-98). The present inventors further hypothesize that the complex bonds initiators directly or indirectly through the inflammatory process to form the protofibril, and that AMPs assist protofibrils to adhere to cell membranes. Likewise small anionic molecules would also stimulate fibril development. For example, heparin and other glycosaminoglycans stimulate the formation of amyloid fibrils from alpha-synuclein in-vitro (Cohlberg J A. et al., 2002. Biochemistry 41:1502-11) in the same way as do small cationic peptides. Therefore down regulating AMPs can be used for preventing age related diseases. Defensins are overexpressed in Alzheimer's disease due to inflammation (Hsiao-Nan et al., Journal of Eurochemistry, 2001, 77, 1027-1035). In high concentrations, defensins, especially alpha-defensins can be cytotoxic to human cells leading to cell death found in Alzheimer's disease, multiple sclerosis and diabetes. Microglia are also activated in Alzheimer's disease, releasing AMPs such as CAP37. The protein Secreted Protein of Streptococcus pyogenes That Inactivates Antibacterial Peptides (SIC; Inga-Maria Frick et al., 2003. J. Biol. Chem. 278:16561-16566) inactivates AMPs and hence can be used as a treatment for Alzheimer's disease and other inflammatory diseases. Defensins attach to complement (especially C1 complement). This complement-AMP attachment is found in Alzheimer's plaques (McGeer, E G. et al., 1994. FEBS Lett. 356:169-73).

As described above, preventing binding of AMPs/AMLs to cognate receptors may be used to inhibit a biological process mediated by binding of the AMP/AML to the receptor. Over 50 AMPs/AMLs and over 20 receptors thereof are involved disease pathogenesis, therefore inhibiting correct target combinations of ligand and receptors is essential for treatment of such diseases. Examples of such AMPs/AMLs and cognate receptors thereof, and the types of diseases which can be treated using this approach are shown in Table 1.

Ample guidance for practicing methods and techniques of the present invention, and for obtaining and utilizing materials employed for practicing the present invention is provided in the literature of the art (refer, for example, to U.S. Patent Application No. 20030044907)

Thus, the present invention enables for the first time relative to the prior art, treatment of any of various diseases, such as psoriasis and tumors, which are associated with biological processes in cells/tissues such as dysregulated growth/differentiation, dysregulated growth/differentiation balance, inflammation, metastasis and angiogenesis using AMPs/AMLs, and/or inhibitors thereof. The present invention also enables for the first time relative to the prior art identification of such AMPs/AMLs, and of such inhibitors.

It is expected that during the life of this patent many relevant drug screening techniques will be developed and the scope of the phrase “method of identifying a compound” is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al, “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Use of Anti-AMP Antibodies for Inhibition of Carcinoma Cell Proliferation and Loss of Substrate-Attachment

### Optimal Treatment Method for Carcinomas Such as Metastatic Malignant Skin Carcinoma

Background: No optimal therapy exists for treatment of metastatic malignant carcinomas, such as metastatic malignant skin carcinoma. An optimal strategy for treating such diseases would be to identify factors involved in inducing carcinoma cell growth and loss of substrate attachment and identifying compounds capable of inhibiting such factors so as to inhibit such growth and loss of substrate attachment. While reducing the present invention to practice, a significant role for AMPs in driving carcinoma proliferation and loss of substrate attachment was identified, and the capacity of anti-AMP antibodies to inhibit such growth and loss of substrate attachment so as to enable optimal treatment of carcinoma, including malignant metastatic skin carcinoma was demonstrated, as described below, thereby overcoming the limitations of the prior art.

Materials and Methods:

Antimicrobial peptides (AMPs): The antimicrobial peptides human beta-defensin-1 and human beta-defensin-2 were obtained from Sigma (Catalogue numbers: D9565 and D9690, respectively).

Antibodies: The anti-human beta-defensin-2 antibody used was polyclonal goat anti-human beta-defensin-2 antibody obtained by immunization with greater than 98% pure recombinant human beta-defensin-2 (GenBank: ACCESSION AAC33549; VERSION AAC33549.1 GI:3510600; DBSOURCE: accession AF040153.1), and affinity chromatography purification of antiserum using an immobilized human beta-defensin-2 matrix.

Thymidine incorporation cell proliferation assay: Cell proliferation was evaluated by measuring [3(H)]-thymidine incorporation into DNA. Cells were pulsed with [3(H)]-thymidine (1 microcurie/mL, ICN, Irvine, Calif.) for 1 hour, at 37 degrees centigrade. After incubation, cells were washed 3 times with PBS, incubated for 15 minutes at room temperature in 5% trichloroacetic acid and solubilized in 1% triton X-100. The radioactivity incorporated into the cells was counted in the [3(H)]-window of a Tricarb liquid scintillation counter. Mean values were determined from measurements of triplicate samples under each experimental condition for each experiment. Thymidine incorporation was determined as number of disintegrations per minute (DPM) per mg of protein.

Experimental Results:

Skin carcinoma cells are significantly stimulated to proliferate by AMPs: In order to investigate the effects of AMPs on malignant keratinocyte growth, epidermal human keratinocyte cell lines were chronically treated with AMPs, and their proliferation was monitored. These cells lines include immortalized (cell lines HaCaT and clone 6), weakly malignant cells (cell lines A-5 and I-5) and highly malignant cells (cell lines II-4 and RT-3). As can be seen in FIG. 1, in all skin epithelial cells exposure to AMPs led to a marked increase in cell proliferation, and to a marked decrease in cell attachment. This data clearly demonstrates that AMPs may be involved in the pathogenesis of carcinomas such as skin carcinoma, both with respect to cellular hyperproliferation as well as with respect to metastasis.

Inhibition of skin carcinoma cell growth and loss of substrate attachment by anti-AMP antibody: Cultured immortalized, moderately malignant or highly malignant human keratinocytes (HaCaT, A-5, and RT-3, respectively) were plated, allowed to attach, incubated in the presence of anti-human beta-defensin-2 antibody at a concentration of 1.0 microgram/ml for 48 hours, and cell proliferation was estimated via [3(H)]-thymidine incorporation assay. As can be seen in FIG. 2, significant inhibition of growth of the malignant keratinocytes was induced by 1.0 microgram/ml of anti-human beta-defensin-2 antibody. The antibody treatment was also observed to result in significantly enhanced substrate-attachment of the cells. These results thereby demonstrate that such anti-AMP antibody treatment can be used to treat carcinomas such as malignant metastatic skin carcinoma

Conclusion: The above-described results clearly demonstrate that AMPs, such as human beta-defensin-1 and human beta-defensin-2, are involved in driving pathogenic proliferation of carcinoma cells, such as metastatic malignant carcinoma cells, and demonstrate for the first time that compounds capable of inhibiting the activity of AMPs, such as anti-AMP antibodies, can be used for optimal treatment of carcinomas, such as malignant metastatic skin carcinomas.

### Example 2

### Use of Anti-AMP Antibodies for Regulation of Skin Cell Proliferation

### Optimal Treatment Method for Diseases Requiring Therapeutic Regulation of Skin Growth, Such as Skin Wounds, Burns, and Skin Tumors

Background: No optimal therapy exists for treatment of various diseases, such as skin wounds and burns, requiring therapeutic regulation of skin growth. An optimal strategy for treating such diseases would be to inhibit the activity of factors involved in preventing skin growth. While reducing the present invention to practice, the capacity of specific concentrations of anti-AMP antibodies to upregulate or downregulate skin growth so as to enable optimal treatment of diseases, such as skin wounds and burns, was demonstrated, as described below, thereby overcoming the limitations of the prior art.

Materials and Methods:

Antibodies: The anti-human beta-defensin-2 antibody used was polyclonal goat anti-human beta-defensin-2 antibody obtained by immunization with greater than 98% pure recombinant human beta-defensin-2 (GenBank: ACCESSION AAC33549; VERSION AAC33549.1 GI:3510600; DBSOURCE: accession AF040153.1), and affinity chromatography purification of antiserum using an immobilized human beta-defensin-2 matrix. The anti-LL-37 antibody used was protein A-purified polyclonal rabbit anti-human LL-37 antibody raised by immunization with the 37 amino acid residue-long LL-37 peptide [amino acid residue coordinates 134-170 of hCAP18/human cathelicidin antimicrobial peptide precursor protein (GenBank: ACCESSION NP_{—}004336; VERSION NP_{—}004336.2 GI:39753970; REFSEQ: accession NM_{—}004345.3)].

Thymidine incorporation cell proliferation assay: Cell proliferation was evaluated by measuring [3(H)]-thymidine incorporation into DNA. Cells were pulsed with [3(H)]-thymidine (1 microcurie/mL, ICN, Irvine, Calif.) for 1 hour, at 37 degrees centigrade. After incubation, cells were washed 3 times with PBS, incubated for 15 minutes at room temperature in 5% trichloroacetic acid and solubilized in 1% triton X-100. The radioactivity incorporated into the cells was counted in the [3(H)]-window of a Tricarb liquid scintillation counter. Mean values were determined from measurements of triplicate samples under each experimental condition for each 10 experiment. Thymidine incorporation was determined as number of disintegrations per minute (DPM) per mg of protein.

Experimental Results:

Concentration-dependent upregulation or downregulation of primary keratinocyte growth by anti-AMP antibodies: To investigate the effects of anti-AMP antibodies on skin growth, cultured primary keratinocytes were treated for 48 hours with antibody against LL-37 (blue bars) at concentrations of 4 (“1×”) or 20 (“5×”) micrograms/ml, or with anti-human beta-defensin-2 antibody (yellow bars) at concentrations of 1 (“1×”) or 5 (“5×”) micrograms/ml, and cell proliferation was measured. As shown in FIG. 3, treatment with 4 or 1 micrograms/ml of anti-LL-37 or human beta-defensin-2 antibody, respectively, resulted in significant induction of keratinocyte proliferation, whereas treatment with 5-fold higher concentrations, 20 or 5 micrograms/ml, respectively, of such antibodies unexpectedly resulted in significant growth inhibition of the keratinocytes.

Conclusion: The above-described results clearly demonstrate for the first time relative to the prior art that antibodies specific for AMPs such as human beta-defensin-2 and LL-37 can be used for positively and negatively regulating skin growth, and hence can be used for optimal treatment of diseases such as those requiring therapeutic skin growth which include, for example, skin wounds and burns.

### Example 3

### Use of Anti-AMP Antibodies for Optimal Treatment of Diseases, Such as Psoriasis, which are Associated with Inflammation, Autoimmunity and/or Skin Cell/Tissue Proliferation/Differentiation Imbalance

Background: Diseases associated with inflammation, autoimmunity and/or skin cell/tissue proliferation/differentiation imbalance include numerous diseases, such as psoriasis and dandruff, for which no optimal therapy exists. Angiogenesis and epithelialization common in psoriatic skin is enhanced by AMPs such as LL-37 (Koczulla, R. et al., 2003. J. Clin. Invest 111:1665-1672; Heilborn, J D. et al., 2003. J Invest Dermatol 120:379-389). An optimal strategy for treating such diseases would be to identify factors involved in dysregulation of skin cell/tissue proliferation/differentiation, and to use compounds capable of inhibiting the activity of such factors to treat such diseases. Such compounds, however, have not been identified. AMPs/AMLs involved in psoriasis include psoriasin, defensins, LL-37, CTACK/CCL27, CCL28, fractalkine, neutrophil gelatinase-associated lipocalin (NGAL) (Exp Dermatol. 2002, 11:584-91). Therefore the present inventors have hypothesized that inhibiting regulating such AMPs/AMLs may be used for treating psoriasis. While reducing the present invention to practice, a method of using anti-AMP antibodies for optimal treatment in a human of a disease associated with inflammation, autoimmunity and/or skin cell/tissue proliferation/differentiation imbalance, such as psoriasis, was demonstrated for the first time, as described below, thereby overcoming the limitations of the prior art.

Materials and Methods:

Antimicrobial peptides (AMPs): The antimicrobial peptide human beta-defensin-2 was employed (Sigma Cat. No. D9690).

Antibodies: The anti-human beta-defensin-2 antibody used was polyclonal goat anti-human beta-defensin-2 obtained by immunization with greater than 98% pure recombinant human beta-defensin-2 (GenBank: ACCESSION AAC33549; VERSION AAC33549.1 GI:3510600; DBSOURCE: accession AF040153.1), and affinity chromatography purification of antiserum using an immobilized human beta-defensin-2 matrix. The anti-LL-37 antibody used was protein A-purified polyclonal rabbit anti-human LL-37 antibody raised by immunization with the 37 amino acid residue-long LL-37 peptide [amino acid residue coordinates 134-170 of hCAP18/human cathelicidin antimicrobial peptide precursor protein (GenBank: ACCESSION NP_{—}004336; VERSION NP_{—}004336.2 GI:39753970; REFSEQ: accession NM_{—}004345.3)].

Three-dimensional organotypic in-vitro skin cultures: Dermal equivalents for organotypic cocultures were prepared with native type I collagen extracted from rat tail tendons with 0.1% acetic acid. The lyophilized collagen was redissolved with 0.1% acetic acid to a final concentration of 4 mg per ml. Eight volumes of ice-cold collagen solution were mixed with 1 volume of 10× Hank's buffered saline followed by neutralization with 2 molar NaOH. One volume of fetal calf serum (FCS) was added together with suspended murine fibroblasts (passages 5-8) and mixed thoroughly resulting in a final concentration of 3.2 mg collagen per ml and 100,000 cells per ml. From this mixture, 2.5 ml aliquots are poured into polycarbonate membrane filter inserts (Falcon no. 3501, Becton Dickinson, Heidelberg, Germany), placed in special deep six well trays (Becton Dickinson) and allowed to solidify at 37 degrees centigrade. Glass rings (24 mm outer, 20 mm inner diameter) were placed onto the gels, to compress them and to provide a flat central area for keratinocyte seeding. The gels were equilibrated with DMEM (Biological Industries, Israel) supplemented with 10% FCS and 50 mg L-ascorbic acid (Sigma) per ml. The next day, 1,000,000 HaCaT human cultured non-malignant keratinocytes (2.5-3.5×10⁵ per square centimeter) were seeded in DMEM supplemented with 10% FCS and 50 mg L-ascorbic acid (Sigma) per ml on the collagen matrix. After submersion in medium and overnight incubation, the cultures were raised to the air-medium interface by lowering the medium level. The cultures were further incubated with medium changes every 2-3 d.

Human in-vivo psoriatic lesion treatment: Anti-LL-37 antibody (100 micrograms/ml) diluted in PBS containing 0.1% BSA, or negative control antibody-free buffer carrier was applied to lesions in a human subject in a blind trial.

Experimental Results:

Correction of skin proliferation/differentiation imbalance by anti-AMP (human beta-defensin-2) antibody: A unique model of 3D organotypic skin co-culture was established in order to investigate the effects of AMPs on proliferation-differentiation imbalance of skin cells/skin, and to investigate the possibility that AMP activity inhibition will correct such imbalance. Primary human keratinocytes were seeded on a mouse fibroblast-containing collagen gel dermal layer equivalent. Within a few weeks, under the growth conditions described above, the 3D-organotypic coculture became organized, mimicking the histological structure of the skin tissue in vivo, including the skin epidermal layers as well as keratin formation. Appropriate proliferation-differentiation of keratinocytes and fibroblasts is a prerequisite for full organization of the organotypic skin co-culture. Any imbalance will lead to inability of the epidermal cells to form a mature fully developed skin equivalent.

In order to examine the effects of AMPs on the growth/differentiation balance of skin cells/skin, the cultured skin was exposed to 20 ng/ml of human beta-defensin-2. As can be seen in FIG. 4b, such exposure to human beta-defensin-2 led to decreased ability of the keratinocytes to form a normal epidermal layer in comparison to the untreated control (FIG. 4a). However, treatment with 1 microgram/ml of anti-human beta-defensin-2 antibody unexpectedly led to significant restoration of the proliferation/differentiation imbalance, as evidenced by the normal histological differentiation of the cultured skin (FIG. 4c).

These results strongly suggested, therefore that treatment with anti-AMP antibody could be used to treat diseases, such as psoriasis and dandruff, which are associated with an imbalance in skin cell/tissue proliferation/differentiation.

While conceiving the present invention, the present invention theorized that cancer is a permanent imbalance between the proliferation/differentiation pathways caused by an imbalance in the proportion of outer membrane defensin-1 (downregulated) and defensin-2 (upregulated), and hence that the presently described method can be used for treating cancer.

Efficient treatment of in-vivo human psoriatic skin lesions by anti-AMP (LL37) antibody: In order to investigate the possibility of using anti-AMP antibodies for treating diseases, such as psoriasis, which are associated with inflammation, autoimmunity and/or an imbalance in skin cell/tissue proliferation/differentiation, in-vivo human psoriatic lesions were treated with anti-AMP (LL-37) antibody. The treatment was performed using a blind trial by topical application on psoriatic lesions daily for 3 days of anti-LL-37 antibody at a concentration of 100 micrograms/ml, and by monitoring the appearance of the lesions after 10 hours and subsequently for a duration 14 days. As control, a non specific antibody was applied on an adjacent lesion in the same subject The treatment solutions applied in a blind trial were identified 10 hours following treatment (data not shown). Treatment with the antibody unexpectedly specifically resulted in significant healing of the treated lesions after only 10 hours. As can be seen in FIGS. 5a-d, healing of the lesions in response to anti-AMP antibody treatment resulted in a significant decrease in inflammation and scaling three days following treatment. The effects of the treatment lasted for at least two weeks following treatment (data not shown). The experiment was repeated 4 times on different lesions giving the same results each time. Conclusion: The above-described results clearly demonstrate for the first time relative to the prior art, treatment of a disease using an anti-AMP antibody. Specifically, the above described results clearly demonstrate for the first time relative to the prior art optimal in-vivo treatment in a human subject of a disease, such as psoriasis, which is associated with inflammation and/or skin cell/tissue proliferation/differentiation imbalance, using anti-AMP antibody.

### Example 4

### Regulation of Gastrointestinal Epithelial Cell Proliferation Using Anti-AMP Antibody

### Optimal Treatment Method for Diseases Associated with Gastrointestinal Cell Hyperproliferation Such as Inflammatory Bowel Diseases, Helicobacter-Associated Gastrointestinal Diseases, and Gastrointestinal Carcinomas

Background: No optimal therapy exists for treatment of diseases associated with dysregulated gastrointestinal epithelial cell proliferation such as inflammatory bowel diseases, Helicobacter-associated gastrointestinal diseases, and gastrointestinal carcinomas. While reducing the present invention to practice, the capacity of anti-AMP antibodies to regulate growth of gastrointestinal epithelial cells so as to enable optimal treatment of diseases associated with dysregulated gastrointestinal cell proliferation, such as inflammatory bowel diseases, Helicobacter-associated gastrointestinal diseases, and gastrointestinal carcinomas, was demonstrated, as described below, thereby overcoming the limitations of the prior art.

Materials and Methods:

Antibodies: The anti-human beta-defensin-2 antibody used was polyclonal goat anti-human beta-defensin-2 antibody obtained by immunization with greater than 98% pure recombinant human beta-defensin-2 (GenBank: ACCESSION AAC33549; VERSION AAC33549.1 GI:3510600; DBSOURCE: accession AF040153.1), and affinity chromatography purification of antiserum using an immobilized human beta-defensin-2 matrix.

Thymidine incorporation cell proliferation assay: Cell proliferation was evaluated by measuring [3(H)]-thymidine incorporation into DNA. Cells were pulsed with [3(H)]-thymidine (1 microcurie/mL, ICN, Irvine, Calif.) for 1 hour, at 37 degrees centigrade. After incubation, cells were washed 3 times with PBS, incubated for 15 minutes at room temperature in 5% trichloroacetic acid and solubilized in 1% triton X-100. The radioactivity incorporated into the cells was counted in the [3(H)]-window of a Tricarb liquid scintillation counter. Mean values were determined from measurements of triplicate samples under each experimental condition for each experiment. Thymidine incorporation was determined as number of disintegrations per minute (DPM) per mg of protein.

Experimental Results:

Significant concentration-dependent negative or positive regulation of gastrointestinal epithelial cell proliferation by anti-AMP (human beta-defensin-2) antibody: In order to investigate the effects of anti-AMP antibodies on proliferation of gastrointestinal epithelial cells/epithelium, cultured Caco2 human gastrointestinal epithelial cells were treated for 48 hours with anti-human beta-defensin-2 antibody at 0.5 or at 1.0 microgram/ml concentration, and cell proliferation was measured via [3(H)]-thymidine incorporation assay. The antibody was unexpectedly uncovered to have significant concentration-dependent regulatory effect on the growth of the cells. As can be seen in FIG. 6, at 1 microgram/ml concentrations of the antibody there was in inhibitory effect on the growth of the gastrointestinal epithelial cells, whereas at the lower concentration of 0.5 microgram/ml, the antibody stimulated increased cell proliferation.

Conclusion: The above-described results clearly demonstrate for the first time relative to the prior art that anti-AMP antibodies can be used for upregulation and downregulation of gastrointestinal epithelial cells. As such, the above described results provide an optimal method for treating gastrointestinal diseases associated with dysregulated growth of gastrointestinal epithelial cells, such as inflammatory bowel diseases, Helicobacter infection-associated diseases, and gastrointestinal carcinomas.

### Example 5

Inhibition of Endothelial Cell Proliferation Using Anti-AMP Antibody

### Optimal Treatment Method for Diseases Associated with Endothelial Hyperproliferation/Angiogenesis and/or Inflammation, Such as Solid Malignancies, Psoriasis, Autoimmune Diseases and Endothelial Tumors

Background: No optimal therapy exists for treatment of diseases associated with endothelial hyperproliferation/angiogenesis and/or inflammation, such as solid malignancies, endothelial tumors, autoimmune diseases and psoriasis. While reducing the present invention to practice, the capacity of anti-AMP antibodies to inhibit growth of endothelial cells/angiogenesis so as to enable optimal treatment of diseases associated with endothelial hyperproliferation/angiogenesis and/or inflammation, such as solid malignancies, psoriasis, autoimmune diseases and endothelial tumors was demonstrated, as described below, thereby overcoming the limitations of the prior art.

Materials and Methods:

Antibodies: The anti-human beta-defensin-2 antibody used was polyclonal goat anti-human beta-defensin-2 antibody obtained by immunization with greater than 98% pure recombinant human beta-defensin-2 (GenBank: ACCESSION AAC33549; VERSION AAC33549.1 GI:3510600; DBSOURCE: accession AF040153.1), and affinity chromatography purification of antiserum using an immobilized human beta-defensin-2 matrix.

Thymidine incorporation cell proliferation assay: Cell proliferation was evaluated by measuring [3(H)]-thymidine incorporation into DNA. Cells were pulsed with [3(H)]-thymidine (1 microcurie/mL, ICN, Irvine, Calif.) for 1 hour, at 37 degrees centigrade. After incubation, cells were washed 3 times with PBS, incubated for 15 minutes at room temperature in 5% trichloroacetic acid and solubilized in 1% triton X-100. The radioactivity incorporated into the cells was counted in the [3(H)]-window of a Tricarb liquid scintillation counter. Mean values were determined from measurements of triplicate samples under each experimental condition for each experiment. Thymidine incorporation was determined as number of disintegrations per minute (DPM) per mg of protein.

Experimental Results:

Significant inhibition of endothelial cell proliferation by anti-AMP (human beta-defensin-2) antibody: In order to investigate the effects of anti-AMP antibodies on endothelial cell proliferation, bovine primary endothelial cells were treated for 48 hours with anti-human beta-defensin-2 antibody at 0.5 or at 1.0 microgram/ml concentration, and proliferation was assessed via [3(H)]-thymidine incorporation assay. Antibody treatment was found to have a significant inhibitory effect on endothelial cell proliferation, particularly at a concentration of 0.5 micrograms/ml, as can be seen in FIG. 7.

Conclusion: The above-described results clearly demonstrate for the first time relative to the prior art that anti-AMP antibodies, such anti-human beta-defensin-2 antibodies, can be used for significantly inhibiting endothelial proliferation/angiogenesis and/or inflammation, and hence can be used for optimal treatment of diseases associated with endothelial hyperproliferation/angiogenesis and/or inflammation, such as solid malignancies, endothelial tumors, autoimmune diseases and psoriasis.

### Example 6

### Cell Culture and Protein Lysate Preparation

Cell Culture and protein lysate preparation: Primary human and murine keratinocytes or cell lines and human and murine fibroblasts are prepared and maintained as described previously (Wertheimer, E. et al., 1993. Nat. Genet. 5:71-73; Spravchikov, N. et al., 2001. Diabetes 50:1627-1635) Keradtinocytes: Briefly, freshly isolated keratinocytes were cultured in Eagle's medium (Biological Industries, Beit Haemek, Israel) with 10% chelexed fetal calf serum (Biological Industries, Beit Haemek, Israel), 1% antibiotics and Ca2+ concentration adjusted to 0.05 mM. After 5 days in culture, in order to induce differentiation, the growth medium was switched to medium containing Ca2+ at defined concentrations ([Ca2+] of 0.05 mM for proliferating phase; 0.12 mM for induction of differentiation; 1.0 mM for terminal differentiation) for 48 hours. After 48 hours, unless indicated otherwise, cells were harvested by scraping into lysis buffer [Phosphate-buffered saline (PBS) containing Triton X-100, 1%; EDTA, 1 mM; sodium fluoride, 10 mM; sodium 200 micromolar orthovanadate; and a protease inhibitor cocktail]. The lysate was microcentrifuged at maximum speed and the Triton-soluble supernatant was further analyzed by SDS-PAGE and immunoblotting. The Triton-insoluble pellet was kept for analysis of cytoskeletal proteins, as described below. Protein concentrations were measured using a modified Lowry assay (Bio-Rad DC Protein Assay Kit).

Preparation of cytoskeletal protein samples for analysis of keratin expression: The Triton-insoluble fraction (pellet) obtained as described above was incubated for 30 minutes in a special lysis buffer containing beta-mercaptoethanol (20%) and SDS (5%). The samples were spun for 30 minutes at maximal speed in a microcentrifuge, and the lysate was further analyzed by SDS-PAGE and Western blot analysis following standard procedures.

### Example 7

### Chemotaxis Assays

Chemotaxis assays Cells (e.g. neutrophils, monocytes, T cells, HEK293; 25 microliters at a density of 1.0-3.0×10⁶ cells/ml) in RPMI medium (Beit Haemek) containing 0.5% BSA (Sigma-Aldrich) are placed on the top of a 96-well ChemoTx disposable chemotaxis apparatus with a 5 micron pore size (Neuroprobe). Tenfold serial dilutions of the tested reagent in RPMI medium with or without 0.5% BSA are placed in the bottom wells of the chamber. The apparatus is incubated for 60-600 min at 37° C. in an atmosphere of 5% carbon dioxide, and the cells migrating at each concentration of chemoattractant is counted with the use of an inverted microscope.

Cells (1×10⁷/mL) are suspended in a buffer containing 0.25% BSA, 145 mM NaCl, 5 mM KCl, 10 mM Na/MOPS, 1 mM CaCl₂, 1 mM MgCl₂, 10 mM glucose, 10 mM HEPES (all from Sigma-Aldrich), pH 7.4, and incubated with 2 micromolar Fura-2-AM (Molecular Probes, Eugene, Oreg.), for 40 min at room temperature. The cells are washed once, resuspended in the buffer containing 0.25% BSA, and are kept at room temperature. Just before use, aliquots of the cells (4×10⁵) are washed and resuspended in 2 ml buffer containing 0.05% BSA in a stirred cuvette at 37° C. Measurement of intracellular Ca2+ concentration and chemotaxis assays are performed as previously described (Maghazachi, A A. et al., 1997. FASEB J. 11:765-774)

### Example 8

### Psoriasis Animal Models

Human Psoriatic Skin-SCID Mouse Transplant Model: Transplantation of human skin onto immunocompromised mice (either congenitally athymic [nude] mice or severe combined immunodeficiency [SCID] mice) provides one of the an approach to the study of psoriasis.

SCID mice (CB-17 strain; Taconic Farms Inc., Germantown, N.Y.) will be used as tissue recipients. Keratomed tissue samples as well as 20 ml blood will be obtained from normal or psoriatic volunteer and cut into 1×1 cm sections. Main blood components involved are Natural Killer cells. Two to four mice will be transplanted bilaterally with each human skin sample, depending on tissue availability. After mice will be anesthetized (sodium pentobarbital; 1.8 mg per 25 gm body weight, i.p.), the dorsal region of each mouse will be shaved bilaterally. Mouse skin will be surgically removed to size, and replaced with the human tissue. The transplanted tissue will be secured to the back of the mouse with absorbable sutures (4-0 Dexon “S”; Davis-Geck, Manati, Puerto Rico). The transplants will be further bandaged with Xeroform petrolatum dressing for 5 days. The animals will be maintained in a pathogen-free environment throughout the preparation and treatment phases. PBMC is isolated from the blood obtained. In some animals Psoriasis is enhanced or maintained by injection of the donors activated PBMC (super antigen) into xenograft. (Smith, T, Nickoloff, B J., 1996. J. Clin. Invest 98:1878-1887).

Antibody screening will be initiated 3 to 5 weeks after transplantation.

Flaky skin (fsn) mouse model: Another model that will be tested is a murine model that express a psoriasiform phenotype i.e., the flaky skin (fsn) mutation. Breeding pairs of CBy.A fsn/J mice (The Jackson Laboratory, Bar Harbor, Me.) will be obtained. As the genetic defect resulting in the flaky skin phenotype is unknown and as homozygous mutant mice are not fertile, the offspring of CBY(FSN/fsn) mice will be used for all experiments. In the CBy.A background, erythrosquamous skin lesions are readily seen at the age of 5-6 weeks, allowing the separation of fsn/fsn mice from their wild-type or heterozygous littermates. For antibody treatment studies, mice will be used between 12 and 16 weeks of age (littermates in most cases), after it has been established that the phenotype remained stable within this time frame.

Animal treatment protocols: Animals will be divided into treatment groups (vehicle plus test reagents) or a control group (vehicle alone). The monoclonal antibodies or inhibitory agents will be delivered topically, intradermal or intraperitoneally in 100 microliters of PBS (6 mg/kg of body weight as an initial concentration used. This will be adjusted according to results. The control mice were treated with PBS alone. Treatment was continued daily for 14 days.

Quantitative Evaluation of Epidermal Thickness: After the treatment phase, mice will be killed and the transplanted human tissue surgically removed and fixed in 3% formalin. After paraffin embedding, one to three 5-micron-thick sections will be cut from each tissue piece, mounted onto microscope slides, and stained with hematoxylin and eosin. The epidermal area will be measured as a function of changes in epidermal thickness per unit length using NIH Image software (National Institutes of Health, Bethesda, Md.). Specifically, randomly chosen tissue section fields will be visualized by light microscopy at ×10 magnification. At this level of magnification, the entire epidermal area of each tissue section is “captured” in equal segments (three to four segments across a typical tissue section), and the area of each segment can be quantified using the NIH Image analysis program. Multiple areas from bilateral transplants on two to four mice per treatment group for each donor will be quantified in this way, to provide 100 or more measurements. The mean epidermal area will be determined from these values. For the Human Psoriatic Skin—SCID Mouse Transplant Model an additional control value will be set; Before transplantation, a small piece of tissue from each donor will be fixed in 3% buffered formalin and used for zero-time assessment of epidermal thickness.

Histology and Immunohistochemical Assessment: Several other histologic characteristics of psoriasis will be followed to evaluate the effectiveness of treatment. This including epidermal hyperplasia, and dermal and/or intra-epidermal infiltration with lymphocytes and neutrophils. For this purpose 5-microm-thick sections will be obtained from each tissue piece, stained with hematoxylin and eosin, and evaluated microscopically.

Statistical Analysis: Statistical significance will be assessed by the paired two-tailed Student's t-test, and P<0.05 will be considered significant. In addition, measurements of epidermal thickness for each group will be analyzed by ANOVA and comparisons between paired groups. The analysis accounts for the correlation between pre-treatment values and post-treatment values for each individual tissue, using a mixed model approach.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and patent applications and sequences identified by their accession numbers mentioned in this specification are herein incorporated in their entirety by reference into the specification, to the same extent as if each individual publication, patent, or patent application or sequence identified by its accession number was specifically and individually indicated to be incorporated herein by reference. In addition, citation or identification of any reference in this application shall not be construed as an admission that such reference is available as prior art to the present invention.

1-99. (canceled)

100. A method of treating a disease in a subject in need thereof, the method comprising providing to the subject a therapeutically effective amount of a compound being capable of decreasing an activity and/or level of an antimicrobial peptide (AMP) and/or AMP-like molecule, thereby treating the disease in the subject in need thereof.

101. The method of claim 100, wherein said compound is selected from the group consisting of:
(a) a molecule capable of binding said AMP and/or AMP-like molecule;
(b) an enzyme capable of cleaving said AMP and/or AMP-like molecule;
(c) an siRNA molecule capable of inducing degradation of an mRNA encoding said AMP and/or AMP-like molecule;
(d) a DNAzyme capable of cleaving an mRNA or DNA encoding said AMP and/or AMP-like molecule;
(e) an antisense polynucleotide capable of hybridizing with an mRNA encoding said AMP and/or AMP-like molecule;
(f) a ribozyme capable of cleaving an mRNA encoding said AMP and/or AMP-like molecule;
(g) a non-functional analogue of at least a functional portion of said AMP and/or AMP-like molecule;
(h) a molecule capable of inhibiting activation or ligand binding of said AMP and/or AMP-like molecule; and
(i) a triplex-forming oligonucleotide capable of hybridizing with a DNA encoding said AMP and/or AMP-like molecule.

102. The method of claim 101, wherein said molecule capable of binding said AMP and/or AMP-like molecule is an antibody or an antibody fragment.

103. The method of claim 100, wherein said AMP and/or AMP-like molecule is a beta-defensin.

104. The method of claim 100, wherein said AMP and/or AMP-like molecule is selected from the group consisting of beta-defensin-1 beta-defensin-2 and LL-37.

105. The method of claim 100, wherein the disease is selected from the group consisting of a tumor, an autoimmune disease, an epithelial disease, a skin disease, a gastrointestinal disease, and an endothelial disease.

106. An article of manufacture comprising packaging material and a pharmaceutical composition, the article of manufacture being identified for treatment of a disease being associated with a biological process in a cell and/or tissue, the biological process being selected from the group consisting of growth, differentiation, inflammation, metastasis and angiogenesis; the pharmaceutical composition comprising a pharmaceutically acceptable carrier and, as an active ingredient, a compound being capable of decreasing an activity and/or level of an antimicrobial peptide (AMP) and/or AMP-like molecule.

107. The article of manufacture of claim 106, wherein said compound is selected from the group consisting of:
(a) a molecule capable of binding said AMP and/or AMP-like molecule;
(b) an enzyme capable of cleaving said AMP and/or AMP-like molecule;
(c) an siRNA molecule capable of inducing degradation of an mRNA encoding said AMP and/or AMP-like molecule;
(d) a DNAzyme capable of cleaving an mRNA or DNA encoding said AMP and/or AMP-like molecule;
(e) an antisense polynucleotide capable of hybridizing with an mRNA encoding said AMP and/or AMP-like molecule;
(f) a ribozyme capable of cleaving an mRNA encoding said AMP and/or AMP-like molecule;
(g) a non-functional analogue of at least a functional portion of said AMP and/or AMP-like molecule; and
(h) a molecule capable of inhibiting activation or ligand binding of said AMP and/or AMP-like molecule; and
(i) a triplex-forming oligonucleotide capable of hybridizing with a DNA encoding said AMP and/or AMP-like molecule.

108. The article of manufacture of claim 107, wherein said molecule capable of binding said AMP is an antibody or an antibody fragment.

109. The article of manufacture of claim 106, wherein said AMP and/or AMP-like molecule is a beta-defensin.

110. The article of manufacture of claim 106, wherein said AMP and/or AMP-like molecule is selected from the group consisting of beta-defensin-1 beta-defensin-2 and LL-37.

111. The article of manufacture of claim 106, wherein said disease is selected from the group consisting of a tumor, an autoimmune disease, an epithelial disease, a skin disease, a gastrointestinal disease, an endothelial disease and a human disease.

112. The article of manufacture of claim 106, wherein said disease is psoriasis or skin carcinoma.

113. A method of treating a disease in a subject in need thereof, the method comprising providing to the subject a therapeutically effective amount of an antimicrobial peptide (AMP) and/or AMP-like molecule, thereby treating the disease in the subject in need thereof.

114. The method of claim 113, wherein said providing to the subject said AMP and/or AMP-like molecule is effected by administering said AMP and/or AMP-like molecule to the subject and/or by expressing said AMP and/or AMP-like molecule in the subject.

115. The method of claim 113, wherein said AMP and/or AMP-like molecule is a beta-defensin.

116. The method of claim 113, wherein said AMP and/or AMP-like molecule is selected from the group consisting of beta-defensin-1, beta-defensin-2 and LL-37.

117. The method of claim 113, wherein the disease is selected from the group consisting of a tumor, an epithelial disease, a skin disease, a gastrointestinal disease and an endothelial disease.

118. An article of manufacture comprising packaging material and a pharmaceutical composition, the article of manufacture being identified for treatment of a disease being associated with a biological process in a cell and/or tissue, said biological process being selected from the group consisting of growth, differentiation, inflammation and angiogenesis; the pharmaceutical composition comprising a pharmaceutically acceptable carrier and, as an active ingredient, an antimicrobial peptide (AMP) and/or AMP-like molecule.

119. The article of manufacture of claim 118, wherein said AMP and/or AMP-like molecule is a beta-defensin.

120. The article of manufacture of claim 118, wherein said AMP and/or AMP-like molecule is selected from the group consisting of beta-defensin-1 beta-defensin-2 and LL-37.

121. The article of manufacture of claim 118, wherein said disease is selected from the group consisting of a tumor, an epithelial disease, a skin disease, a gastrointestinal disease and an endothelial disease.
